SEQUENCE LISTING (16 sequences)

SEQ ID NO: 1 - RNA, 21 nt, Artificial sequence
  Strand of a siRNA
  misc_feature (20)..(21): n is an overflowing deoxythymidine
gacucagcugccccauccann

SEQ ID NO: 2 - RNA, 21 nt, Artificial sequence
  Strand of a siRNA
  misc_feature (20)..(21): n is an overflowing deoxythymidine
uggauggggcagcugagucnn

SEQ ID NO: 3 - RNA, 19 nt, Artificial sequence
  Strand of a siRNA
gacucagcugccccaucca

SEQ ID NO: 4 - RNA, 19 nt, Artificial sequence
  Strand of a siRNA
uggauggggcagcugaguc

SEQ ID NO: 5 - DNA, 10661 bp, artificial sequence
  cDNA sequence corresponding to the androgen receptor-encoding mRNA in humans
  cDNA (1)..(10661)
  ref <308>: DATABASE ACCESSION NUMBER: NM_000044.3
  ref <309>: DATABASE ENTRY DATE: 2016-07-09
  ref <313>: RELEVANT RESIDUES IN SEQ ID NO: (1)..(10661)
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaacaaaaaaaaaaagcaaaaacaaaacaaaaaataagccaaaaaaccttgctagtgttttttcctcaaaaataaataaataaataaataaatacgtacatacatacacacatacatacaaacatatagaaatccccaaagaggccaatagtgacgagaaggtgaaaattgcaggcccatggggagttactgattttttcatctcctccctccacgggagactttattttctgccaatggctattgccattagagggcagagtgaccccagagctgagttgggcaggggggtggacagagaggagaggacaaggagggcaatggagcatcagtacctgcccacagccttggtccctgggggctagactgctcaactgtggagcaattcattatactgaaaatgtgcttgttgttgaaaatttgtctgcatgttaatgcctcacccccaaacccttttctctctcactctctgcctccaacttcagattgactttcaatagtttttctaagacctttgaactgaatgttctcttcagccaaaacttggcgacttccacagaaaagtctgaccactgagaagaaggagagcagagatttaaccctttgtaaggccccatttggatccaggtctgctttctcatgtgtgagtcagggaggagctggagccagaggagaagaaaatgatagcttggctgttctcctgcttaggacactgactgaatagttaaactctcactgccactaccttttccccacctttaaaagacctgaatgaagttttctgccaaactccgtgaagccacaagcaccttatgtcctcccttcagtgttttgtgggcctgaatttcatcacactgcatttcagccatggtcatcaagcctgtttgcttcttttgggcatgttcacagattctctgttaagagcccccaccaccaagaaggttagcaggccaacagctctgacatctatctgtagatgccagtagtcacaaagatttcttaccaactctcagatcgctggagcccttagacaaactggaaagaaggcatcaaagggatcaggcaagctgggcgtcttgcccttgtcccccagagatgataccctcccagcaagtggagaagttctcacttccttctttagagcagctaaaggggctacccagatcagggttgaagagaaaactcaattaccagggtgggaagaatgaaggcactagaaccagaaaccctgcaaatgctcttcttgtcacccagcatatccacctgcagaagtcatgagaagagagaaggaacaaagaggagactctgactactgaattaaaatcttcagcggcaaagcctaaagccagatggacaccatctggtgagtttactcatcatcctcctctgctgctgattctgggctctgacattgcccatactcactcagattccccacctttgttgctgcctcttagtcagagggaggccaaaccattgagactttctacagaaccatggcttctttcggaaaggtctggttggtgtggctccaatactttgccacccatgaactcagggtgtgccctgggacactggttttatatagtcttttggcacacctgtgttctgttgacttcgttcttcaagcccaagtgcaagggaaaatgtccacctactttctcatcttggcctctgcctccttacttagctcttaatctcatctgttgaactcaagaaatcaagggccagtcatcaagctgcccattttaattgattcactctgtttgttgagaggatagtttctgagtgacatgatatgatccacaagggtttccttccctgatttctgcattgatattaatagccaaacgaacttcaaaacagctttaaataacaagggagaggggaacctaagatgagtaatatgccaatccaagactgctggagaaaactaaagctgacaggttccctttttggggtgggatagacatgttctggttttctttattattacacaatctggctcatgtacaggatcacttttagctgttttaaacagaaaaaaatatccaccactcttttcagttacactaggttacattttaataggtcctttacatctgttttggaatgattttcatcttttgtgatacacagattgaattatatcattttcatatctctccttgtaaatactagaagctctcctttacatttctctatcaaatttttcatctttatgggtttcccaattgtgactcttgtcttcatgaatatatgtttttcatttgcaaaagccaaaaatcagtgaaacagcagtgtaattaaaagcaacaactggattactccaaatttccaaatgacaaaactagggaaaaatagcctacacaagcctttaggcctactctttctgtgcttgggtttgagtgaacaaaggagattttagcttggctctgttctcccatggatgaaaggaggaggattttttttttcttttggccattgatgttctagccaatgtaattgacagaagtctcattttgcatgcgctctgctctacaaacagagttggtatggttggtatactgtactcacctgtgagggactggccactcagacccacttagctggtgagctagaagatgaggatcactcactggaaaagtcacaaggaccatctccaaacaagttggcagtgctcgatgtggacgaagagtgaggaagagaaaaagaaggagcaccagggagaaggctccgtctgtgctgggcagcagacagctgccaggatcacgaactctgtagtcaaagaaaagagtcgtgtggcagtttcagctctcgttcattgggcagctcgcctaggcccagcctctgagctgacatgggagttgttggattctttgtttcatagctttttctatgccataggcaatattgttgttcttggaaagtttattatttttttaactcccttactctgagaaagggatattttgaaggactgtcatatatctttgaaaaaagaaaatctgtaatacatatatttttatgtatgttcactggcactaaaaaatatagagagcttcattctgtcctttgggtagttgctgaggtaattgtccaggttgaaaaataatgtgctgatgctagagtccctctctgtccatactctacttctaaatacatataggcatacatagcaagttttatttgacttgtactttaagagaaaatatgtccaccatccacatgatgcacaaatgagctaacattgagcttcaagtagcttctaagtgtttgtttcattaggcacagcacagatgtggcctttccccccttctctcccttgatatctggcagggcataaaggcccaggccacttcctctgccccttcccagccctgcaccaaagctgcatttcaggagactctctccagacagcccagtaactacccgagcatggcccctgcatagccctggaaaaataagaggctgactgtctacgaattatcttgtgccagttgcccaggtgagagggcactgggccaagggagtggttttcatgtttgacccactacaaggggtcatgggaatcaggaatgccaaagcaccagatcaaatccaaaacttaaagtcaaaataagccattcagcatgttcagtttcttggaaaaggaagtttctacccctgatgcctttgtaggcagatctgttctcaccattaatctttttgaaaatcttttaaagcagtttttaaaaagagagatgaaagcatcacattatataaccaaagattacattgtacctgctaagataccaaaattcataagggcagggggggagcaagcattagtgcctctttgataagctgtccaaagacagactaaaggactctgctggtgactgacttataagagctttgtgggtttttttttccctaataatatacatgtttagaagaattgaaaataatttcgggaaaatgggattatgggtccttcactaagtgattttataagcagaactggctttccttttctctagtagttgctgagcaaattgttgaagctccatcattgcatggttggaaatggagctgttcttagccactgtgtttgctagtgcccatgttagcttatctgaagatgtgaaacccttgctgataagggagcatttaaagtactagattttgcactagagggacagcaggcagaaatccttatttctgcccactttggatggcacaaaaagttatctgcagttgaaggcagaaagttgaaatacattgtaaatgaatatttgtatccatgtttcaaaattgaaatatatatatatatatatatatatatatatatatatatatagtgtgtgtgtgtgttctgatagctttaactttctctgcatctttatatttggttccagatcacacctgatgccatgtacttgtgagagaggatgcagttttgttttggaagctctctcagaacaaacaagacacctggattgatcagttaactaaaagttttctcccctattgggtttgacccacaggtcctgtgaaggagcagagggataaaaagagtagaggacatgatacattgtactttactagttcaagacagatgaatgtggaaagcataaaaactcaatggaactgactgagatttaccacagggaaggcccaaacttggggccaaaagcctacccaagtgattgaccagtggccccctaatgggacctgagctgttggaagaagagaactgttccttggtcttcaccatccttgtgagagaagggcagtttcctgcattggaacctggagcaagcgctctatctttcacacaaattccctcacctgagattgaggtgctcttgttactgggtgtctgtgtgctgtaattctggttttggatatgttctgtaaagattttgacaaatgaaaatgtgtttttctctgttaaaacttgtcagagtactagaagttgtatctctgtaggtgcaggtccatttctgcccacaggtagggtgtttttctttgattaagagattgacacttctgttgcctaggacctcccaactcaaccatttctaggtgaaggcagaaaaatccacattagttactcctcttcagacatttcagctgagataacaaatcttttggaattttttcacccatagaaagagtggtagatatttgaatttagcaggtggagtttcatagtaaaaacagcttttgactcagctttgatttatcctcatttgatttggccagaaagtaggtaatatgcattgattggcttctgattccaattcagtatagcaaggtgctaggttttttcctttccccacctgtctcttagcctggggaattaaatgagaagccttagaatgggtggcccttgtgacctgaaacacttcccacataagctacttaacaagattgtcatggagctgcagattccattgcccaccaaagactagaacacacacatatccatacaccaaaggaaagacaattctgaaatgctgtttctctggtggttccctctctggctgctgcctcacagtatgggaacctgtactctgcagaggtgacaggccagatttgcattatctcacaaccttagcccttggtgctaactgtcctacagtgaagtgcctggggggttgtcctatcccataagccacttggatgctgacagcagccaccatcagaatgacccacgcaaaaaaaagaaaaaaaaaattaaaaagtcccctcacaacccagtgacacctttctgctttcctctagactggaacattgattagggagtgcctcagacatgacattcttgtgctgtccttggaattaatctggcagcaggagggagcagactatgtaaacagagataaaaattaattttcaatattgaaggaaaaaagaaataagaagagagagagaaagaaagcatcacacaaagattttcttaaaagaaacaattttgcttgaaatctctttagatggggctcatttctcacggtggcacttggcctccactgggcagcaggaccagctccaagcgctagtgttctgttctctttttgtaatcttggaatcttttgttgctctaaatacaattaaaaatggcagaaacttgtttgttggactacatgtgtgactttgggtctgtctctgcctctgctttcagaaatgtcatccattgtgtaaaatattggcttactggtctgccagctaaaacttggccacatcccctgttatggctgcaggatcgagttattgttaacaaagagacccaagaaaagctgctaatgtcctcttatcattgttgttaatttgttaaaacataaagaaatctaaaatttcaaaaaa

SEQ ID NO: 6 - DNA, 1651 bp, artificial sequence
  cDNA sequence corresponding to the histone H2AX-encoding mRNA in humans
  cDNA (1)..(1651)
  ref <308>: DATABASE ACCESSION NUMBER: NM_002105.2
  ref <309>: DATABASE ENTRY DATE: 2017-10-09
  ref <313>: RELEVANT RESIDUES IN SEQ ID NO: (1)..(1651)
acagcagttacactgcggcgggcgtctgttctagtgtttgagccgtcgtgcttcaccggtctacctcgctagcatgtcgggccgcggcaagactggcggcaaggcccgcgccaaggccaagtcgcgctcgtcgcgcgccggcctccagttcccagtgggccgtgtacaccggctgctgcggaagggccactacgccgagcgcgttggcgccggcgcgccagtgtacctggcggcagtgctggagtacctcaccgctgagatcctggagctggcgggcaatgcggcccgcgacaacaagaagacgcgaatcatcccccgccacctgcagctggccatccgcaacgacgaggagctcaacaagctgctgggcggcgtgacgatcgcccagggaggcgtcctgcccaacatccaggccgtgctgctgcccaagaagaccagcgccaccgtggggccgaaggcgccctcgggcggcaagaaggccacccaggcctcccaggagtactaagagggcccgcgccgcggccggccgccaggcctccccatgccaccacaaaggcccttttaagggccaccaccgccctcatggaaagagctgagccgcttcagactgcggggcaagcgggccgcggctcccttcccctcccctcccctcgcccgccttcgccgcccggcctcgagtccccgcccgcccccgctcccgtcccgcaccgcctgccgcgtcggcctcgggccctgccctgtccgccgtccgccctccggtagggttcgggccttccggatgcggcttgggcgctcttcggggacctccgtggcgcggaagacccgagcctgccggggggaggccggcggcgccgcacctgcccgcctcggcgttcgtgactcagccgccccatcccgagtcgctaaggggctgcggggaggccgcagcaccttctggaagacttggccttccgctctgacgcagggccgaggtgggcagtccaggccgagaggccggcggccctgaaggtgagtgaggccctcggcagctgcagccggggtgtctggtacccccccggcgtggtgcttagcccaggactttcagacgcggccgctggccgggaggctttggtgggagagacgcgatcgccgatttcggtctggcgccccttctgcggccgggacccaggcctttcacatcagctctccctccatcttcattcataggtctgcgctggggccgggacgaagcacttggtaacaggcacatcttcctcccgagtgactgcctcctaggaggacatttaggggagggcagaggcctgcagtttggcttcacggctggctatgtggacagcaagagtcgttttcgcggaagccgactggcagccaggcctgtcgggccccccgacgccgccccatttcccttccagcaaactcaactcggcaatccaagcacctagataccagcacaagtcggttaatccctgtctggactgagcctccgttggcttctgaactggaattctgcagctaacccttccacgactagaaccttaggcattggggagttttagatggactaattttattaaaggattgttttttttttaaaaaaaaaaaaaaaaaaaaaaaaaaaaaaaaaaaaaaaaaaaaaaaaaaaaaaaaaaaa

SEQ ID NO: 7 - RNA, 21 nt, Artificial sequence
  Strand of a siRNA
  misc_feature (20)..(21): n is an overflowing deoxythymidine
uagcaaugacgaaugcguann

SEQ ID NO: 8 - RNA, 21 nt, Artificial sequence
  Strand of a siRNA
  misc_feature (20)..(21): n is an overflowing deoxythymidine
uacgcauucgucauugcuann

SEQ ID NO: 9 - RNA, 23 nt, Artificial sequence
  Strand of a siRNA
  misc_feature (22)..(23): n is an overflowing deoxythymidine
gacucagcugccccauccacgnn

SEQ ID NO: 10 - RNA, 23 nt, Artificial sequence
  Strand of a siRNA
  misc_feature (22)..(23): n is an overflowing deoxythymidine
cguggauggggcagcugagucnn

SEQ ID NO: 11 - RNA, 21 nt, Artificial sequence
  Strand of a siRNA
  misc_feature (20)..(21): n is an overflowing deoxythymidine
uccccaagcccaucguagann

SEQ ID NO: 12 - RNA, 21 nt, Artificial sequence
  Strand of a siRNA
  misc_feature (20)..(21): n is an overflowing deoxythymidine
ucuacgaugggcuuggggann

SEQ ID NO: 13 - RNA, 21 nt, Artificial sequence
  Strand of a siRNA
  misc_feature (20)..(21): n is an overflowing deoxythymidine
aaugauacgaucgaguuccnn

SEQ ID NO: 14 - RNA, 21 nt, Artificial sequence
  Strand of a siRNA
  misc_feature (20)..(21): n is an overflowing deoxythymidine
ggaacucgaucguaucauunn

SEQ ID NO: 15 - RNA, 21 nt, Artificial sequence
  Strand of a siRNA
  misc_feature (20)..(21): n is an overflowing deoxythymidine
cuggaauucugcagcuaacnn

SEQ ID NO: 16 - RNA, 21 nt, Artificial sequence
  Strand of a siRNA
  misc_feature (20)..(21): n is an overflowing deoxythymidine
guuagcugcagaauuccagnn

(19) United States
(12) Patent Application Publication
(10) Pub. No.: US 2021/0292755 A1
(43) Pub. Date: Sep. 23, 2021

(54) NOVEL DOUBLE-STRANDED OLIGONUCLEOTIDES FOR THE TREATMENT OF CANCER

(71) Applicant: SELEXEL, TOULOUSE CEDEX 1 (FR)

(72) Inventors: Florence CABON, GOYRANS (FR); Hilary BROOKS, SAINT ORENS DE GAMEVILLE (FR); Stéphanie DELMAS, FROUZINS (FR); Maud CHUSSEAU, SAUBENS (FR)

(21) Appl. No.: 16/345,610

(22) PCT Filed: Oct. 27, 2017
(86) PCT No.: PCT/FR2017/052976

§ 371 Date: Apr. 26, 2019

(30) Foreign Application Priority Data
Oct. 27, 2016 (FR) 16/60441
Aug. 8, 2017 (FR) 17/57591

(51) Int. Cl. C12N 15/113; A61K 45/06; A61P 35/00

(52) U.S. Cl. CPC C12N 15/113; A61P 35/00; A61K 45/06

(57) ABSTRACT

Disclosed is a double-stranded oligonucleotide for the use thereof in the prevention and/or treatment of cancer, in association with a therapy that causes damage to DNA or prevents the repair thereof. Also disclosed is a double-stranded oligonucleotide for the use thereof in the prevention and/or treatment of cancer, with the exception of androgen-dependent prostate cancer.

### BACKGROUND OF THE INVENTION

### Field of the Invention

The present invention relates to a bispecific double-stranded oligonucleotide, at the same time targeting the androgen receptor and histone H2AX.

### Description of the Related Art

Cells of our organism undergo daily a large number of lesions of the DNA, provoked either by their metabolism which produces for example reactive species of oxygen, or by their environment, in particular by the ionizing radiation or UV, agents physical or chemical, including certain drugs (https://en.wikipedia.org/wiki/DNA_damage_naturally_occurring)). Cells developed several mechanisms of verification and repair during evolution to maintain the integrity of the genome. When DNA damage occurs in a normal cell, tumor suppressor proteins, such as p53, orchestrate the blockage of cell division and the activation of repair mechanisms. Most lesions are normally repaired. Beyond a certain threshold of DNA damage, however, the cell is no longer viable, enters apoptosis and dies.

In the same situation, cancer cells accumulate a large number of DNA lesions because their DNA repair mechanisms are frequently deficient and/or because they are more resistant to apoptosis than normal cells. The threshold of damage to DNA leading to cell death is higher in a tumor cell than in a normal cell.

DNA lesions occur in a context where DNA is not linear but is compacted by nucleosomes consisting of histones. Some histones play a role of structure, others, like H2AX, are key in the functioning or the repair of the genome. H2AX is a histone expressed in all the cells of the body. The phosphorylation of H2AX, then noted γH2AX, for example by proteins such as DNA-PK (DNA-dependent protein kinase), ATM (mutated in ataxia-telangiectasia) or ATR (ataxia telangiectasia and Rad3-related), is a major event and early in DNA repair, which allows for the recruitment and assembly of many other proteins needed to repair the lesion.

The detection of γH2AX, in particular by specific antibodies, is frequently used as a sensitive and quantitative indicator of the presence of DNA lesions. H2AX can also be acetylated on lysine 36 by CBP/P300 and on threonine 101, and these two post-translational modifications allow cells to survive ionizing radiation, independently of the phosphorylation of H2AX. Inhibiting the expression of H2AX in a cell, particularly in a cancer cell, is a method that prevents the repair of DNA damage. However, due to the multiplicity of DNA repair mechanisms, inhibition of H2AX expression alone is not sufficient to induce cell death (Yuan and Chen, 2010).

Radiation therapy or certain chemotherapies have been developed to treat cancers by increasing the number of DNA lesions to exceed the threshold compatible with cell viability, threshold beyond which the apoptosis of the cancer cell is triggered. These treatments work by inducing DNA damage and/or preventing the repair of lesions present in cancer cells. Radiation therapy is now essential in oncology since it is programmed in two thirds of treatment regimens, either alone or associated with surgery and/or chemotherapy. These treatments induce DNA damage, and in particular double-strand breaks (DSB).

Cytotoxic chemotherapy aims to block the divisions of cancer cells and/or to induce their apoptosis. Most of these treatments lead to the accumulation of DNA lesions in the cell, either directly due to their mode of action that creates new lesions, either by preventing DNA repair, either by maintaining the cell at a cell cycle stage wherein the repair processes are poorly active. However, the many genome-testing and repair mechanisms that attempt to repair DNA damage reduce the effectiveness of these chemotherapy or radiation treatments. Thus, there is a real need to improve the effectiveness of therapies inducing DNA damage or preventing their repair.

By preventing the repair of DNA damage, the inhibition of H2AX thus provides a means of enhancing cell death induced by radiotherapy or chemotherapy treatments inducing DNA damage or preventing their repair.

The androgen receptor (AR) is a transcription factor whose activity is regulated by its ligands. AR is expressed in both men and women by many cell types in most tissues and organs and in the hematopoietic system. AR regulates the expression of very many genes involved, for example, in proliferation, migration, survival, invasion, production of growth factors. Due to these properties, a deregulation of the expression or the functioning of AR, for example and in a non-limiting way by overexpression, mutation, alternative splicing, post-translational modification or protein partner change, is described in the initiation, the maintenance and progression of various carcinomas, sarcomas, myelomas and leukemias, for example and in a non-limiting manner, certain cancers of the prostate, breast, ovary, bladder, liver, colon, stomach, adrenal and salivary glands, thymus, thyroid, uterus, peritoneum, pancreas, testes, kidney, skin, brain, nasopharynx, head and neck, meninges, lungs, testes, bones (Munoz et al., 2015).

Castration, which prevents the production of AR ligands and therefore the transcriptional activity of AR, is a therapeutic strategy whose efficacy has been clinically demonstrated in prostate cancer. However, the effectiveness of these treatments is transient and patients relapse systematically. There is therefore a real need for these cancers to improve the effectiveness of treatments targeting the AR signaling pathway.

Although AR is expressed in most cells of the body, and the deregulation of its expression or function is described in many cancers other than those of the prostate, the inhibition of the AR signaling pathway has not been shown to be sufficient to treat previously developed cancers in humans other than prostate cancer. Thus, the proliferation of other cancer cells, for example from breast tumors, melanomas or glioblastoma, that express AR at levels ranging from very low to high, is not affected by inhibition of this pathway, as shown in the examples below.

The present invention is based on the unexpected results of the inventors according to which certain particular double-stranded oligonucleotides are bi-specific, since they simultaneously and specifically inhibit the expression of histone H2AX and that of the androgen receptor, which effectively inhibits the proliferation and survival of cells.

This effect of simultaneous inhibition of AR and H2AX can be enhanced by combining with a chemotherapy or radiation treatment that increases the number of DNA lesions or prevents their repair.

Micro RNAs (miRNAs) are small double-stranded RNAs of about twenty nucleotides encoded by the genome of all eukaryotic organisms. After transcription and maturation, they are loaded into a protein complex: RNA Induced Silencing Complex (RISC). One of the two strands hybridizes, generally imperfectly in mammals, with one or more messenger RNAs (mRNAs), called target mRNA, which they induce either the cleavage, leading to the degradation of the mRNA, or the inhibition of protein translation. Hybridization with one or more mRNAs of nucleotides 2 to 8 inclusive of miRNA (5′ to 3′ sense of the guide strand also called antisense strand), a region called nucleation region or “seed region”, is of particular importance to trigger the mechanism of RNA interference. In the same cell, a miRNA can hybridize with several target mRNAs and regulate their stability or translation. No computer or other tool can accurately predict the mRNAs regulated by a given miRNA (Gorski et al., 2017).

Interfering RNAs or Small Interfering RNAs, or siRNAs, are synthetic double-stranded oligoribonucleotides that when introduced into cells mimic the action of miRNAs, hybridize with their target mRNA, and trigger the RNA interference mechanism. Like miRNAs, they can hybridize incompletely with other mRNAs and computer tools do not accurately predict among those many possible incomplete hybridizations those that interfere with mRNA and disrupt function. These so-called off-target effects are generally undesirable because they can produce potentially toxic biological effects that, if not predicable, must therefore be evaluated experimentally. Using multiple siRNAs simultaneously multiplies the number of potential off-target effects and therefore the risk of toxicity. To choose the sequence of a siRNA for inhibiting the expression of a gene of interest, there are many computer tools. These tools are designed so that the chosen siRNA hybridizes with only one mRNA so as to avoid off-target effects.

The mechanism of action and the pharmacokinetic and pharmacodynamic properties of siRNAs are not comparable to any other type of oligonucleotide such as ribozymes, morpholinos, triple helix oligonucleotides, antisense RNA or antisense DNA oligonucleotides (ODNs).

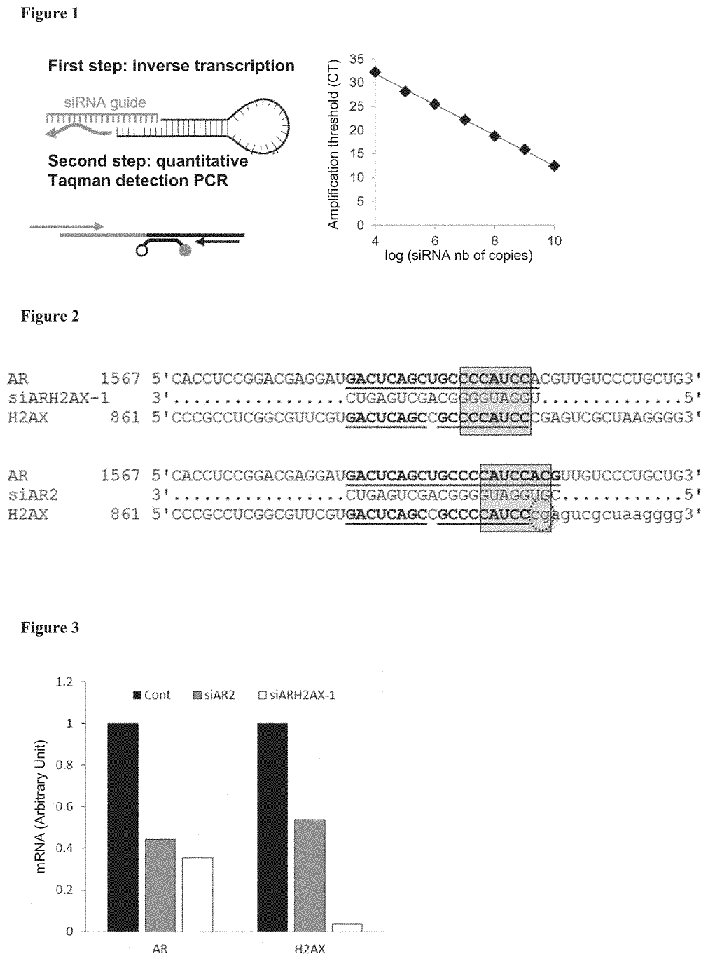

The present invention is based on the unexpected results of the inventors according to which certain particular siRNAs described in Table 1, which hybridize perfectly with the androgen receptor-encoding mRNA and which inhibit its expression, are bi-specific because they also partially hybridize with the H2AX-encoding mRNA as described in FIG. 1 and also specifically inhibit its expression.

### SUMMARY OF THE INVENTION

The present invention aims to provide a means for simultaneously inhibiting the expression of the androgen receptor and H2AX by the use of a bispecific siRNA.

By simultaneously inhibiting the androgen receptor, key in many cancerous processes, especially in proliferation and resistance to apoptosis, and histone H2AX, key in the repair of DNA damage, the use of a bispecific AR-H2AX siRNA, inhibiting both AR and H2AX, has a specific advantage over a siRNA targeting only AR, or a siRNA targeting only H2AX. The use of a single bispecific siRNA is preferable to the combination of two siRNAs, one targeting AR and the other targeting H2AX, to limit the potentially toxic off-target effects of such a combination, and to facilitate the development of clinical and industrial applications of this use.

In a first aspect, the invention relates to a composition comprising at least one bispecific siRNA, defined as wholly or partially hybridizing with both an androgen receptor-encoding mRNA and an H2AX-encoding mRNA, such that this hybridization induces the degradation of these mRNAs or inhibits their translation, said siRNA being chosen from siARH2AX-1, SEQ ID NO: 1 and SEQ ID NO: 2 or siARH2AX-1b, SEQ ID NO: 3 and SEQ ID NO: 4, as described in Table 1, for its use in the prevention and/or treatment of cancer, wherein said siRNA is used in combination with a therapy resulting in DNA damage or preventing their repair.

By partial hybridization is meant the hybridization of a sufficient number of nucleotides of the bispecific siRNA of the invention with the sequence of the androgen receptor mRNA or with that of H2AX to cause either cleavage or inhibition of the translation of the mRNA with which it hybridizes, which leads to a decrease in the expression of the protein encoded by mRNA, a decrease that can be measured, for example, by a Western blot immunoassay or indirect immunofluorescence immunoassay. For example, this hybridization may involve 7, 8, 9, 10, 11, 12, 13, 14, 15, 16, 17, 18, 19 nucleotides, or more if the siRNA has more nucleotides, for example 20, 21, 22 or 23. For example, in the sense of the invention, the percentage of hybridization can be between 30 and 95%.

According to the invention, said prevention and/or said treatment is carried out in a human patient or in a mammal.

The siRNA sequences of the present invention are shown in Table 1. The expression “at least 75% identity with a sequence” in Table 1 means 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%, in particular 79%, 81%, 84%, 86%, 90%, 95% and 99%.

All siRNAs whose sequence is 100% identical to that of the siRNAs shown in Table 1 hybridize 100% with a region of the androgen receptor-encoding mRNA transcribed from exon 1. Thus, all these siRNAs inhibit the expression of all forms of androgen receptor splicing expressed in cancer cells, including those partially or completely deleted from the ligand binding domain, such as, for example, AR-V7 variants of the receptor expressed in some cancers.

Advantageously, the siRNA guide strand nucleation region siARH2AX-1 and siARH2AX-1b is also perfectly complementary to the H2AX-encoding mRNA.

Chemical Modifications

According to the invention and in all its aspects, said bispecific AR-H2AX siRNA may have chemical modifications on one and/or the other strand, on one or more nucleotides located at the 3′ or 5′ terminal ends, and/or on one or more nucleotides constituting the internal skeleton. For example, said chemical modifications are located on the ribose and/or the base and/or the phosphoric acid. Said chemical modifications comprise, for example, at least one substitution of the 2′-OH group of the ribose with a 2′-O-methyl RNA (2′OMe) or 2′-O-methoxyethyl (2′MOE) or 2′-fluoro (2′F) or 2′-fluoro-β-arabinonucleotide (FANA) group, an alkylation of oxygen in 2′ into aminoethyl-, guanidinoethyl-, cyanoethyi- or allyl, replacing the phosphodiester group with a phosphorothioate, an alkylation or a thiloation of one or more nucleotides of the oligonucleotide, replacing a ribonucleotide with a deoxyribonucleotide, or replacing of a nucleotide by a Locked Nucleic Acid (LNA).

In a particular aspect, said bispecific AR-H2AX siRNA is devoid of chemical modification.

In a particular aspect, said siRNA is devoid of chemical modification, and comprises two deoxynucleotides overflowing at the 3′ end, in particular two desoxythymidines.

In another particular aspect, said siRNA is devoid of chemical modification, and does not comprise two deoxynucleotides overflowing at the 3′ end, in particular two desoxythymidines. According to the invention and in all its aspects, said bispecific AR-H2AX siRNA is formulated in a composition that allows its penetration into tumor cells.

Vectorization Agents and Addressing Molecules

In a particular aspect, said composition contains a vectorization agent. According to the invention, a vectorization agent is an agent that facilitates the penetration of a siRNA into the cells of the organism. This agent may be chemically conjugated to either strand of siRNA, or noncovalently associated with siRNA. These include, for example, lipids, polymers, peptides, dendrimers, simple or complex sugars, polyethylenimine derivatives, nanoparticles, magnetic spheres, or inorganic or organic nanostructures.

In a particular aspect, said composition contains an addressing molecule. According to the invention, an addressing molecule is a molecule addressing the siRNA towards a particular cell type, for example endothelial cells or cancer cells. An addressing molecule is not intended to penetrate the siRNA inside the cell or to penetrate with the siRNA but to increase its concentration to the outer membrane of the cell of interest. For example and non-exhaustively, an addressing molecule may be an aptamer, an antibody, a transferrin, an RGD peptide, the ligand of a receptor, this addressing molecule interacting or binding to a molecule expressed at the targeted cell surface, such as for example and without limitation a receptor, an integrin, a membrane antigen such as for example the Prostate Specific Membrane Antigen (PSMA) or the CD36 receptor.

In a particular aspect of the invention, this targeting molecule is a CD36 receptor ligand, for example oxidized LDLs, or hexarelin peptide or a long chain fatty acid (more than 16 carbons), or a mixture of these components two by two or three to three.

In a preferred aspect of the invention, the abovementioned composition contains oxidized LDLs in a weight:weight ratio of 1 siRNA for 0.01 to 10 oxidized LDLs and preferentially 0.1 to 1, or the hexarelin peptide, in a weight:weight ratio of 1 siRNA for 0.01 to 10 hexarelin, preferably 0.1 to 1.

The addressing molecule may be either associated with siRNA without a covalent bond, or covalently conjugated to either strand of siRNA, or incorporated into a vectorization agent, for example a nanoparticle or a liposome containing siRNA, so as to address the vectorization agent to the target cell or tissue.

In a particular aspect, said composition contains neither a vectorization agent nor an addressing molecule.

In a particular aspect, said composition contains a vectorization agent but no addressing molecule.

In a particular aspect, said composition contains an addressing molecule but no vectorization agent. The addressing molecule is in particular a CD36 ligand, and more particularly oxidized LDLs or hexarelin.

In a particular aspect, said composition contains a vectorization agent and an addressing molecule. The addressing molecule is in particular a CD36 ligand, and more particularly oxidized LDLs or hexarelin.

Local, Locoregional or Systemic Administration

According to the invention and in all its aspects, said composition containing said bispecific AR-H2AX siRNA is formulated to be administered locally or in loco-regional manner, in particular by intratumoral administration, in single dose or by repeated injections, intermittently or continuously by the use of an external or implantable pump, or any other device, or biodegradable compound allowing a slow and continuous release of the composition in the vicinity or in the tumor to be treated (intratumoral administration). According to the invention, the continuous mode of administration aims to keep the concentration of the siRNA substantially constant throughout the entire period of administration of the siRNA in the tissue if it is a local or loco-regional administration or in blood and peripheral tissues if it is a systemic administration. The expression “maintain substantially constant” means that the concentration of siRNA in the blood and peripheral tissues may vary slightly depending on the metabolism of the individual receiving said composition.

By continuous is meant the uninterrupted administration of the composition for a period of 1 day to several months, for example 1 day, 2 days, 3 days, 4 days, 5 days, 6 days, 7 days, 8 days, 9 days, 10 days, 11 days, 12 days, 13 days, 14 days, 15 days, 3 weeks, 1 month, 2 months, 3 months, 4 months, 5 months, 6 months, 9 months, 1 year, 2 years.

In a preferred aspect of the invention, the siRNA delivered in a continuous mode of delivery is delivered without interruption for a period of time greater than or equal to 2 days.

In a preferred aspect of the invention, the siRNA delivered according to a continuous administration mode is delivered without the administration being interrupted beyond the time necessary to recharge or exchange the device delivering the siRNA, for example 4 hours, for a duration of administration ranging from 2 days to 1 year, for example 2 days, 3 days, 4 days, 5 days, 6 days, 7 days, 8 days, 9 days, 10 days, 11 days, 12 days, 13 days, 14 days, 15 days, 3 weeks, 1 month, 2 months, 3 months, 4 months, 5 months, 6 months, 9 months, 1 year.

In a preferred aspect of the invention, the siRNA delivered in a continuous mode of administration is delivered in successive cycles, interrupted by a period without treatment ranging from more than 24 hours to a few weeks, each cycle being defined by continuous administration without interruption greater than the time required to recharge or exchange the device delivering the siRNA, for example 4 hours, and for a duration of administration ranging from 2 days to 1 month.

According to the invention, by intermittent administration is meant any mode of administration in which the period of administration of the product (for example, 10 seconds, 1 minute, 1 hour, 2 hours, 4 hours . . . ) is followed by a period during which the product is not administered (for example, 1 hour, 2 hours, 6 hours, 24 hours, 1 week . . . ). It may, for example and without limitation, be a bolus administration, single or repeated over time or a slow infusion over a period of a few hours, leading over time to a significant variation in the concentration of the product in the tissue or organism.

Cancer mortality frequently results from the dissemination of cancer cells to secondary sites, forming metastases. The systemic administration of bispecific siARH2AX siRNA, which distributes them in several tissues and organs, is therefore advantageous for treating cancers that are metastasized or likely to become so. In the following, by systemic is meant that the siRNA is conveyed in the body to act at a distance from the place where it is administered, as opposed to a local or loco-regional administration, in particular as opposed to an intratumoral administration. The systemic distribution in the body is obtained by any method that results in a passage of siRNA in extracellular fluids such as blood, lymph or cerebrospinal fluid, that the compound containing the siRNA is ingested (orally), or injected (parenterally), or through the skin or mucous membranes. The systemic administration can be carried out by administering a single or repeated dose of said composition, intermittently or else by the use of an external or implantable pump, or any other device, or biodegradable compound allowing a slow and continuous release of said composition, such that the bispecific siARH2AX siRNA is systemically transported via the extracellular fluids in the organism. In a particular aspect, said mode of systemic administration is chosen from the subcutaneous, intravenous, intraperitoneal, intramuscular, intradermal, intranasal, intravaginal, intrarectal, sublingual, intrathecal, intracerebral, oral administration mode, and is especially subcutaneous.

In a preferred aspect, said mode of administration is continuous, subcutaneously.

Buffer and Cations

In a particular aspect, the siARH2AXs according to the invention are administered systemically or locally by being formulated in an acidic pH buffer solution.

A buffer solution according to the present invention provides the pH stability of the siRNA dilution solution. Examples of such buffers are given in Table 3.

In a preferred aspect of the invention, the pH of the buffer solution is acid, ranging from pH 3 to pH 7, preferably from pH 5 to pH 6.5 and preferentially to pH 6.

In a preferred aspect of the invention the buffer solution is a citrate or histidine buffer at pH 6. In a particular aspect, the siARH2AX siRNAs according to the invention are administered systemically or locally in an acidic pH buffer solution supplemented with cations derived from inorganic or organic salts. These cations within the sense of the invention are not constituents of the buffer solution, they are not intended to ensure a buffer effect, but they are added to this buffer solution. These cations are for example and without limitation polyamines, especially putrescine, and/or spermidine, and/or spermine, and/or salts whose cation is chosen from metal cations such as for example Zn²⁺, CO²⁺, Cu²⁺, Mn²⁺, Ca²⁺, Mg²⁺, Fe²⁺, the counterion being of any nature, for example a chloride, nitrate, sulfate or carbonate ion. In a preferred aspect of the invention, the buffer solution contains MgCl₂, ZnCl₂, MnCl₂, or a two-by-two mixture of these salts, or a mixture of the three salts.

In a particular aspect, whether used alone or in combination, the concentration of each cation is from 0.02 mM to 200 mM, preferably from 0.05 to 100 mM and preferably from 1 to 50 mM.

In a preferred aspect of the invention, the cations are added to a buffer solution which is a citrate buffer or a histidine buffer. In a preferred aspect of the invention, the pH of this solution is 6.

Doses

In a particular aspect, said composition is formulated for a mode of administration of bispecific siARH2AX siRNA at a therapeutically effective dose, and in particular at doses of 0.005 mg/kg/day to 30 mg/kg/day, in particular 0.01 mg/kg/day to 10 mg/kg/day. “0.005 mg/kg/day to 30 mg/kg/day” means all doses ranging from 0.005 mg/kg/day to 30 mg/kg/day, e.g. 0.008; 0.01; 0.05; 0.1; 0.5; 1.0; 1.5; 10.0; 10.5; 14.0; 14.5; 20; 20.5; 25; 25.5; 29.5 mg/kg/day.

Association with Treatment Inducing DNA Damage or Preventing their Repair

In a particular aspect of the invention, said composition is used in combination with a treatment inducing DNA lesions or preventing their repair and said treatment being chosen from radiotherapy and/or chemotherapy.

Radiotherapy refers to a method of locoregional treatment of cancers, using radiation to destroy cancer cells by blocking their ability to multiply. The technique and the modalities of radiotherapeutic treatment, in particular the doses of radiation delivered, are adapted according to the cancer and the patient. There are three main radiotherapy techniques: external radiotherapy, curietherapy and vector metabolic radiotherapy. Each of them has its indications according to the type of tumor and its location.
External radiotherapy is the best known and the most used. The radiation source, outside the patient, produces high energy X-ray beams and electron beams.
Curietherapy, also called brachytherapy, is a local radiotherapy by implantation of radioactive grains or needles. Such a therapy is especially used in the treatment of prostate cancers.
Vector metabolic radiotherapy is a radiotherapy technique based on oral administration or injection of a radio-pharmaceutical agent labeled with a radioelement. The radioactive isotope will bind preferentially to the diseased target cells. For example iodine 131 binds to the thyroid. The metastatic bone pains can thus be treated, for example, by injection of samarium 153, liquid strontium chloride-labeled with strontium 894, or chloride-radium 223.

Chemotherapy refers to a drug treatment (cytostatic and antineoplastic chemotherapeutic agents) against cancer.

In a particular aspect, said chemotherapy resulting in DNA lesions or preventing their repair is carried out using at least one chemotherapeutic agent chosen from: alkylating agents, anti-metabolite agents, cytotoxic antibiotics, topoisomerase I inhibitors, topoisomerase II inhibitors, anti-tumor antibiotics, genotoxic agents, PARP inhibitors (poly(ADP-ribose) polymerase), microtubule-forming and dissociation inhibiting agents, such as vinca-alkaloids, taxanes or epothilones,

In a particular aspect, a chemotherapeutic agent is chosen in particular from:

Arabinosylcytosine, Bleomycin, Busulfan, Capecitabine, Carboplatin, Carmustine, Chlorambucil, Cisplatin, Cladribine, Cyclophosphamide, Dacarbazine, Daunorubicin, Doxorubicin, Epirubicin, Etoposide, Floxuridine, Fludarabine, Fluorouracil and in particular 5-Fluorouracil, Gemcitabine, Hydroxyurea, Idarubicin, Ifosfamide, Iniparib, Irinotecan, Lomustine, Mechlorethamine, Melphalan, Mercaptopurine and in particular 6-Mercaptopurine, Methotrexate, Mitomycin C, Mitoxantrone HCl, Mustine, Niraparib, Olaparib, Oxaliplatin, Procarbazine, Rubitecan, Rucaparib, Streptozocin, Talazopanib, Thioguanine and in particular 6-Thioguanine, Topotecan, Veliparib, Actinomycin D, Amsacrine, Anthracyclines, Camptothecin, Epipodophyllotoxin, Plicamycin, Temozolomide, Vincristine, Vinblastine, Vinorelbine, Paclitaxel, Docetaxel, Cabazitaxel, Taxanes, Epothilones.

In a particular aspect, said therapy resulting in DNA lesions or preventing their repair and the administration of said composition containing at least one siAR-H2AX bispecific siRNA are simultaneous or successive. The term “successive” is understood according to the invention of the expression “separated, spread over time”.

In a particular aspect, the invention also relates to a combination product containing:
an siRNA chosen from: siARH2AX-1, siARH2AX-1b and
at least one chemotherapeutic agent causing DNA damage or preventing its repair for simultaneous or sequential use in the prevention and/or treatment of cancer.

In one aspect, the invention also relates to a combination product containing:
an siRNA chosen from: siARH2AX-1, siARH2AX-1b and
at least one radiopharmaceutical agent which causes DNA lesion, for simultaneous or sequential use in the prevention and/or treatment of cancer.

In one aspect, the invention also relates to a combination product formulated for intermittent or continuous local administration containing:
an siRNA chosen from: siARH2AX-1, siARH2AX-1b and
a curietherapy treatment that causes DNA damage, for simultaneous or sequential use in the prevention and/or treatment of cancer.

In one particular aspect, the cancer is a primary tumor, or a metastasis of a primary tumor.

In one particular aspect, the cancer is adrenocortical, esophageal, gastric, basal cells, thyroid or uterine, astrocytoma, glioblastoma, oligodendroglioma, meningioma, thymoma, lymphoma, melanoma, or non-melanoma skin cancer, leukemia, mesothelioma, cholangiocarcinoma, myeloma, gastrointestinal cancer, bladder, breast, cervical, head and neck, non-small cell lung, ovary, pancreas, prostate, testis, thymus, kidney, salivary gland, endometrium, anus, colon, appendix, mouth, bronchi and/or upper airways, bile duct, nasal and paranasal cavity, brain, heart, stomach, liver, throat, tongue, lips, nasopharynx, esophagus, bones, parathyroid, penis, pleura, lung, rectum, adrenal gland, urethra, vagina, gallbladder, vulva, colon adenocarcinoma, rectum, desmoid tumor, nasopharyngeal fibroma, sarcoma, osteosarcoma, leimyosarcoma, chondrosarcoma, liposarcoma, rhabdomyosarcoma, pheochromocytoma or metastasis of these cancers developing in other organs.

In another aspect of the invention, said composition comprising at least one bispecific siRNA, said siRNA hybridizing wholly or partially with both an androgen receptor-encoding mRNA and an H2AX-encoding mRNA, such that hybridization induces the degradation of these mRNAs or inhibits their translation, said siRNA being chosen from siRNA siARH2AX-1, SEQ ID NO: 1 and SEQ ID NO: 2 or siARH2AX-1b, SEQ ID NO: 3 and SEQ ID NO: 4 as described in Table 1, is used alone for its use in the prevention and/or treatment of cancer, in particular adrenocortical, esophageal, gastric, basal cells, thyroid or uterine, astrocytoma, glioblastoma, oligodendroglioma, meningioma, thymoma, lymphoma, melanoma, or non-melanoma skin cancer, leukemia, mesothelioma, cholangiocarcinoma, myeloma, gastrointestinal cancer, bladder, breast, cervical, head and neck, non-small cell lung, ovary, pancreas, prostate, testis, thymus, kidney, salivary gland, endometrium, anus, colon, appendix, mouth, bronchi and/or upper airways, bile duct, nasal and paranasal cavity, brain, heart, stomach, liver, throat, tongue, lips, nasopharynx, esophagus, bones, parathyroid, penis, pleura, lung, rectum, adrenal gland, urethra, vagina, gallbladder, vulva, colon adenocarcinoma, rectum, desmoid tumor, nasopharyngeal fibroma, sarcoma, osteosarcoma, leimyosarcoma, chondrosarcoma, liposarcoma, rhabdomyosarcoma, pheochromocytoma or metastasis of these cancers developing in other organs.

According to the invention, said prevention and/or said treatment is carried out in a human patient or in a mammal.

In a particular aspect, said bispecific AR-H2AX siRNA can have chemical modifications on one and/or the other strand, on one or more nucleotides located at the 3′ or 5′ terminal ends, and/or on one or more nucleotides constituting the internal skeleton. Examples of chemical modifications can be found in the section entitled “Chemical modifications”.

In a particular aspect, said bispecific AR-H2AX siRNA is devoid of chemical modification.

In a particular aspect, said bispecific AR-H2AX siRNA is devoid of chemical modification and comprises two deoxynucleotides overflowing at the 3′ end, in particular two deoxythymidines.

In a particular aspect, said bispecific AR-H2AX siRNA is devoid of chemical modification and does not comprise two deoxynucleotides overflowing at the 3′ end, in particular two deoxythymidines.

In a particular aspect, said composition contains neither a vectorization agent nor an addressing molecule or contains a vectorization agent but no addressing molecule or contains an addressing molecule but no vectorization agent or contains a vectorization agent and an addressing molecule. Examples of addressing agents can be found in the section entitled “Vectorization agents and addressing molecules”

The addressing molecule may be either associated with siRNA without a covalent bond, or covalently coupled with either siRNA strand, or incorporated into a vectorization agent, for example a nanoparticle or a liposome containing siRNA, so as to address the vectorization agent to the target cell or tissue.

In a particular aspect, said composition containing bispecific siARH2AX siRNA is formulated for a systemic, local, or loco-regional administration, in single dose or repeated intermittently or continuously.

In a particular aspect, the siARH2AXs according to the invention are administered systemically or locally by being formulated in an acidic pH buffer solution. Examples of such buffers are given in Table 3.

In a particular aspect, the buffer solution is a citrate or histidine buffer.

In a preferred aspect, the acidic pH buffer solution is supplemented or not with cations from inorganic or organic salts. Examples of cations can be found in the section entitled “Buffer and cations”.

The concentration of each cation is from 0.02 mM to 200 mM, preferably from 0.05 to 100 mM and preferably from 1 to 50 mM.

In a preferred aspect of the invention, the cations are added to a buffer solution which is a citrate buffer or a histidine buffer.

In a particular aspect, said composition is formulated for a mode of administration of siRNA siRNA bispecific at a therapeutically effective dose, and in particular at doses of 0.005 mg/kg/day to 30 mg/kg/day, in particular 0.01 mg/kg/day to 10 mg/kg/day. “0.005 mg/kg/day to 30 mg/kg/day” means all doses ranging from 0.005 mg/kg/day to 30 mg/kg/day, e.g. 0.008; 0.01; 0.05; 0.1; 0.5; 1.0; 1.5; 10.0; 10.5; 14.0; 14.5; 20; 20.5; 25; 25.5; 29.5 mg/kg/day.

In another aspect, the present invention also relates to compositions containing at least one siRNA selected from siRNA: siARH2AX-1, SEQ ID NO: 1 and SEQ ID NO: 2 or siARH2AX-1b, SEQ ID NO: 3 and SEQ ID NO: 4, as described in Table 1, said siRNA being in an acidic pH buffer solution. Examples of such buffers are given in Table 3.

In a particular aspect, said composition is formulated for a systemic, local or loco-regional administration mode, in single dose or repeated intermittently or continuously.

In a particular aspect, the buffer solution is a citrate or histidine buffer.

In a preferred aspect, the acidic pH buffer solution is supplemented or not with cations from inorganic or organic salts. Examples of cations can be found in the section entitled “Buffer and cations”.

The concentration of each cation is from 0.02 mM to 200 mM, preferably from 0.05 to 100 mM and preferably from 1 to 50 mM.

In a preferred aspect of the invention, the cations are added to a buffer solution which is a citrate buffer or a histidine buffer.

In a particular aspect, the present invention also relates to compositions containing at least one siRNA selected from siRNA: siARH2AX-1, SEQ ID NO: 1 and SEQ ID NO: 2 or siARH2AX-1b siRNA, SEQ ID NO: 3 and SEQ ID NO: 4. said siRNA being in a buffer solution at acidic pH, said buffer solution being in particular a citrate or histidine buffer, said buffer solution being supplemented with cations originating from inorganic or organic salts, in particular from a salt whose cation is chosen from polyamines, in particular chosen from spermine, spermidine or putrescine, or in particular from a salt whose cation is chosen from metal cations, in particular chosen from salts of zinc, cobalt, copper, manganese, calcium, magnesium or iron, in particular of manganese, zinc, magnesium, alone or in combination two by two or three to three.

In a particular aspect, said bispecific AR-H2AX siRNA can have chemical modifications on one and/or the other strand, on one or more nucleotides located at the 3′ or 5′ terminal ends, and/or on one or more nucleotides constituting the internal skeleton. Examples of chemical modifications can be found in the section entitled “Chemical modifications”.

In a particular aspect, said bispecific AR-H2AX siRNA is devoid of chemical modification.

In a particular aspect, said bispecific AR-H2AX siRNA is devoid of chemical modification and comprises two deoxynucleotides overflowing at the 3′ end, in particular two deoxythymidines.

In a particular aspect, said bispecific AR-H2AX siRNA is devoid of chemical modification and does not comprise two deoxynucleotides overflowing at the 3′end, in particular two deoxythymidines.

In a particular aspect, said composition contains neither a vectorization agent nor an addressing molecule or contains a vectorization agent but no addressing molecule or contains an addressing molecule but no vectorization agent or contains a vectorization agent and an addressing molecule. Examples of addressing agents can be found in the section entitled “Vectorization agents and addressing molecules”

The addressing molecule may be either associated with siRNA without a covalent bond, or covalently coupled with either siRNA strand, or incorporated into a vectorization agent, for example a nanoparticle or a liposome containing siRNA, so as to address the vectorization agent to the target cell or tissue.

In a particular aspect, said composition is used in combination with a treatment inducing DNA lesions or preventing their repair and said treatment being chosen from radiotherapy and/or chemotherapy. Examples of radiotherapy or chemotherapeutic agents can be found in the section entitled “association with treatment inducing DNA damage or preventing its repair”.

In a particular aspect, said composition is formulated for a mode of administration of siRNA siRNA bispecific at a therapeutically effective dose, and in particular at doses of 0.005 mg/kg/day to 30 mg/kg/day, in particular 0.01 mg/kg/day to 10 mg/kg/day. “0.005 mg/kg/day to 30 mg/kg/day” means all doses ranging from 0.005 mg/kg/day to 30 mg/kg/day, e.g. 0.008; 0.01; 0.05; 0.1; 0.5; 1.0; 1.5; 10.0; 10.5; 14.0; 14.5; 20; 20.5; 25; 25.5; 29.5 mg/kg/day.

TABLE 1

| | | SEQ ID | |
|---|---|---|---|
| siRNA | composed of: | NO: | Séquence 5′- 3′ |
| siARH2AX-1 | siARH2AX-1 Strand 1, the | 1 | GACUCAGCUGCCCCAUCCA[dT] |
| | sequence of which is: | | [dT] |
| | SEQ ID NO: 1 or a | | |
| | sequence having at least | | |
| | 75% identity with said | | |
| | SEQ ID NO: 1 | | |
| | and siARH2AX-1 Strand 2, | 2 | UGGAUGGGGCAGCUGAGUC[dT] |
| | the sequence of which is: | | [dT] |
| | SEQ ID NO: 2 or a | | |
| | sequence having at least | | |
| | 75% identity with said | | |
| | SEQ ID NO: 2 | | |
| siARH2AX-1b | siARH2AX-1 Strand 1b, | 3 | GACUCAGCUGCCCCAUCCA |
| | the sequence of which is: | | |
| | SEQ ID NO: 3 or a | | |
| | sequence having at least | | |
| | 75% identity with said | | |
| | SEQ ID NO: 3 | | |
| | and siARH2AX-1 Strand | 4 | UGGAUGGGGCAGCUGAGUC |
| | 2b, the sequence of which | | |
| | is: SEQ ID NO: 4 or a | | |
| | sequence having at least | | |
| | 75% identity with said | | |
| | SEQ ID NO: 4 | | |

For each of the sequences (SEQ ID NO) numbered from 1 to 4 of Table 1:
The first column indicates the name of the siRNA.
The second column indicates the composition of the siRNA, consisting of the combination of a oligonucleotide strand type 1 (respectively 1b) or an oligonucleotide whose sequence has at least 75% identity with this oligonucleotide strand type 1 (respectively 1b),
and a oligonucleotide strand type 2 (respectively 2b) or an oligonucleotide whose sequence has at least 75% identity with this oligonucleotide strand type 2 (respectively 2b).
The third column indicates the numbering of the oligonucleotide as filed.
The fourth column indicates the sequence in the 5′ to 3′ orientation. The notation [dT][dT] is used to indicate the presence of two overflowing deoxythymidines.

When the siRNAs shown in Table 1 consist of two single-stranded oligonucleotides whose sequence is 100% identical to that shown in the Table, the target sequence (i.e., the sequence of the mRNA to which the siRNA guide strand hybridizes) of these siRNA is present in humans in all the mRNAs encoding the androgen receptor, whether this receptor is wild, that it exhibits splicing variations leading to the partial deletion or total hormone binding domain (variant AR-V7 for example) or that it has one of the mutations described in prostate cancer.

TABLE 2

| Cells | Cancer | AR mRNA (A.U.) |
|---|---|---|
| 22RV1 | Prostate | 100,000 |
| C4-2 | Prostate | 75,786 |
| LNCaP | Prostate | 50,000 |
| MDA-MB-453 | Breast | 18,946 |
| U87-MG | Glioblastoma | 182 |
| PC3 | Prostate | 35 |
| WM266-4 | Skin | 32 |
| MDA-MB-231 | Breast | 2 |

For each of the cell lines used in the examples, the second column indicates the tissue origin of the cells, the level of AR-encoding mRNA was measured by RT-qPCR in these lines and normalized by that of the cyclophilin A-encoding mRNA (delta CT method). The results, given in the third column, are expressed in arbitrary units, the level measured in the 22RV1 cells being considered as 100,000.

TABLE 3
Main buffers

| Buffer | pH area | pKa 25° C. |
|---|---|---|
| maleate (pK1) | 1.2-2.6 | 1.97 |
| phosphate (pK1) | 1.7-2.9 | 2.15 |
| CABS | 10.0-11.4 | 10.7 |
| piperidine | 10.5-12.0 | 11.12 |
| glycine (pK1) | 2.2-3.6 | 2.35 |
| citrate (pK1) | 2.2-6.5 | 3.13 |
| glycylglycine (pK1) | 2.5-3.8 | 3.14 |
| malate (pK1) | 2.7-4.2 | 3.4 |
| formate | 3.0-4.5 | 3.75 |
| citrate (pK2) | 3.0-6.2 | 4.76 |
| succinate (pK1) | 3.2-5.2 | 4.21 |
| acetate | 3.6-5.6 | 4.76 |
| propionate | 3.8-5.6 | 4.87 |
| malate (pK2) | 4.0-6.0 | 5.13 |
| pyridine | 4.9-5.9 | 5.23 |
| piperazine (pK1) | 5.0-6.0 | 5.33 |
| cacodylate | 5.0-7.4 | 6.27 |
| succinate (pK2) | 5.5-6.5 | 5.64 |
| MES | 5.5-6.7 | 6.1 |
| citrate (pK3) | 5.5-7.2 | 6.4 |
| maleate (pK2) | 5.5-7.2 | 6.24 |
| histidine | 5.5-7.4 | 1.70, 6.04, 9.09 |
| bis-tris | 5.8-7.2 | 6.46 |
| phosphate (pK2) | 5.8-8.0 | 7.2 |
| ethanolamine | 6.0-12.0 | 9.5 |
| ADA | 6.0-7.2 | 6.59 |
| carbonate (pK1) | 6.0-8.0 | 6.35 |
| ACES | 6.1-7.5 | 6.78 |
| PIPES | 6.1-7.5 | 6.76 |
| MOPSO | 6.2-7.6 | 6.87 |
| imidazole | 6.2-7.8 | 6.95 |
| BIS-TRIS propane | 6.3-9.5 | 6.80, 9.00 |
| BES | 6.4-7.8 | 7.09 |
| MOPS | 6.5-7.9 | 7.14 |
| HEPES | 6.8-8.2 | 7.48 |
| TES | 6.8-8.2 | 7.4 |
| MOBS | 6.9-8.3 | 7.6 |
| DIPSO | 7.0-8.2 | 7.52 |
| TAPSO | 7.0-8.2 | 7.61 |
| triethanolamine (TEA) | 7.0-8.3 | 7.76 |
| pyrophosphate | 7.0-9.0 | 0.91, 2.10, 6.70, 9.32 |
| HEPPSO | 7.1-8.5 | 7.85 |
| POPSO | 7.2-8.5 | 7.78 |
| tricine | 7.4-8.8 | 8.05 |
| hydrazine | 7.5-10.0 | 8.1 |
| glycylglycine (pK2) | 7.5-8.9 | 8.25 |
| Trizma (tris) | 7.5-9.0 | 8.06 |
| EPPS, HEPPS | 7.6-8.6 | 8 |
| BICINE | 7.6-9.0 | 8.26 |
| HEPBS | 7.6-9.0 | 8.3 |
| TAPS | 7.7-9.1 | 8.4 |
| 2-amino-2-methyl-1,3-propanediol | 7.8-9.7 | 8.8 |
| (AMPD) | | |
| TABS | 8.2-9.6 | 8.9 |
| AMPSO | 8.3-9.7 | 9 |
| taurine (AES) | 8.4-9.6 | 9.06 |
| borate | 8.5-10.2 | 9.23, 12.74, 13.80 |
| CHES | 8.6-10.0 | 9.5 |
| 2-amino-2-methyl-1-propanol (AMP) | 8.7-10.4 | 9.69 |
| glycine (pK2) | 8.8-10.6 | 9.78 |
| ammonium hydroxide | 8.8-9.9 | 9.25 |
| CAPSO | 8.9-10.3 | 9.6 |
| carbonate (pK2) | 9.5-11.1 | 10.33 |
| methylamine | 9.5-11.5 | 10.66 |
| piperazine (pK2) | 9.5-9.8 | 9.73 |
| CAPS | 9.7-11.1 | 10.4 |
| phosphate (pK3) | | 12.33 |

### REFERENCES

Gorski, S. A., J. Vogel, and J. A. Doudna. 2017. RNA-based recognition and targeting: sowing the seeds of specificity. Nat Rev Mol Cell Biol 18:215-228.
Munoz, J., J. J. Wheler, and R. Kurzrock. 2015. Androgen receptors beyond prostate cancer: an old marker as a new target. Oncotarget 6:592-603.
Yuan, J., and J. Chen. 2010. MRE11-RAD5O-NBS1 complex dictates DNA repair independent of H2AX. J Biol Chem 285:1097-1104.

### BRIEF DESCRIPTION OF THE DRAWINGS

The invention will be better illustrated by the following examples and figures. The following examples are intended to clarify the purpose of the invention and to illustrate advantageous embodiments, but in no case are intended to restrict the scope of the invention.

### LEGEND OF FIGURES

FIG. 1: Schematic representation of the method of quantification of a siRNA by RT qPCR. Example of reference range.

FIG. 2: Comparison of the complementarity of the siRNA sequences of the invention and siAR2 siRNA with the androgen receptor-encoding mRNA in humans and that encoding H2AX.

The positions indicated correspond respectively to those of the human androgen receptor mRNA (AR, accession number: NM_00044.3, SEQ ID NO: 5) and H2AX mRNA in humans (accession number: NM_002105.2, SEQ ID NO: 6).

The underlined bases are those which are perfectly complementary to those of the guide strand of the indicated siRNAs. The siRNAs indicated are composed of oligonucleotides 100% identical to those described in Table 1.

The gray boxes indicate the nucleation region (seed region), i.e., the nucleotides ranging from 2 to 8 inclusive siRNA guide strand (5′→3′ direction) and their complementarity with the mRNA encoding H2AX.

FIG. 3: Quantification by RT-qPCR of AR- and H2AX-encoding mRNAs 48 h after transfection in C4-2 cells of control siRNA (Cont, black bars), siAR2 (gray bars) or siARH2AX-1 (white bars). The level of expression of each mRNA is expressed relative to that measured in cells transfected with a control siRNA.

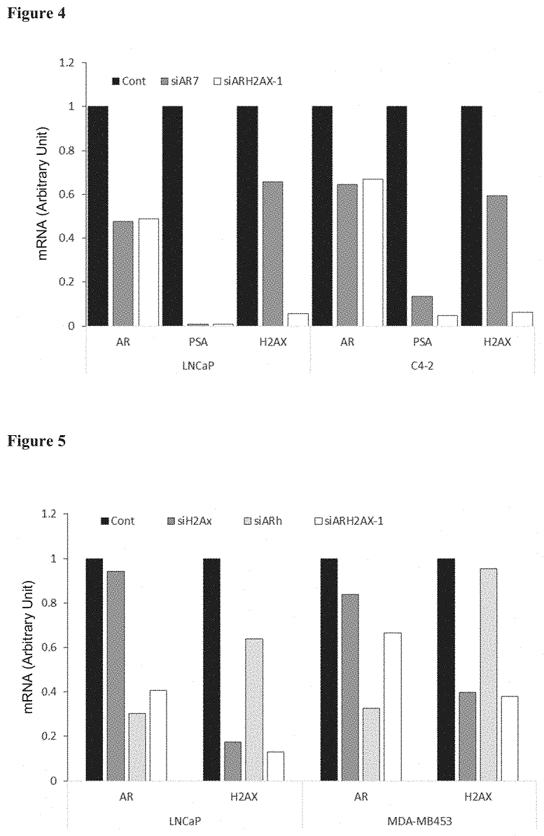

FIG. 4: Quantification by RT-qPCR of the AR-, PSA- and H2AX-encoding mRNAs 72 h after transfection in the LNCaP or C4-2 Cells of a control siRNA (cont, black bars), siAR7 (dark gray bars), or siARH2AX-1 (white bars). The level of expression of each mRNA is expressed relative to that measured in cells transfected with a control siRNA.

FIG. 5: Quantification by RT-qPCR of AR- and H2AX-encoding mRNAs 72 h after transfection in LNCaP or MDA MB-453 cells of a control siRNA (cont, black bars), SiH2AX (dark gray bars), siARh (bars light gray), or siARH2AX-1 (white bars). For each cell type, the level of expression of each mRNA is expressed relative to that measured in cells transfected with a control siRNA.

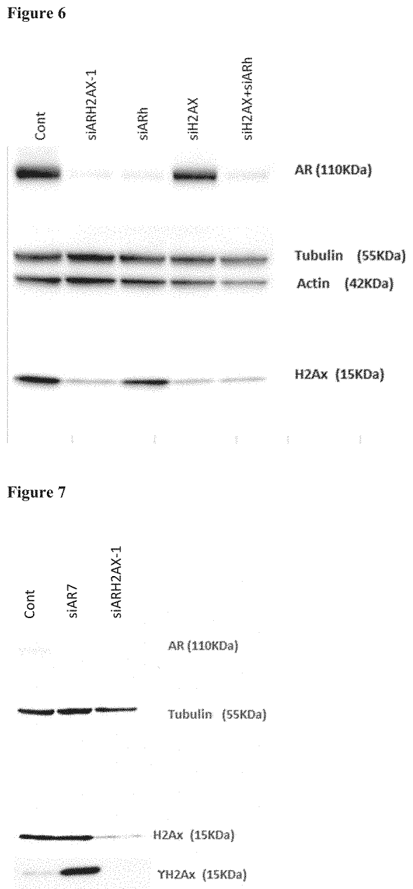

FIG. 6: Immuno detection by western blot of the expression of AR and H2AX 72 h after transfection of C4-2 cells by one of the following siRNAs: Control (Cont), siARH2AX-1, siRNA, siH2AX, or a mixture of both siRNA siH2AX and siARh.

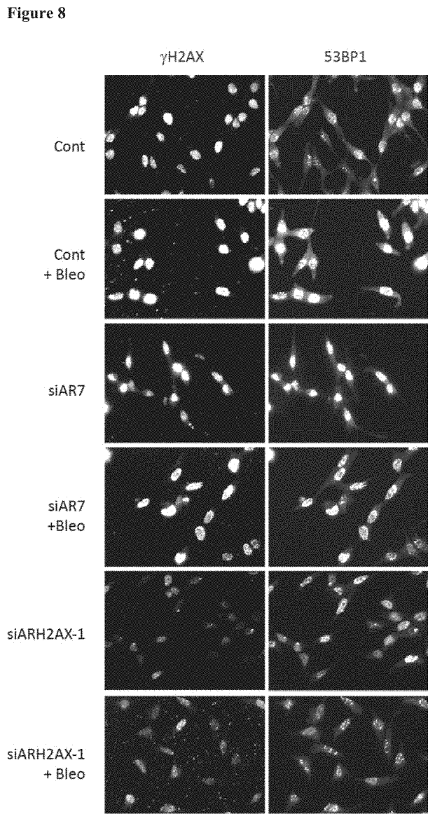

FIG. 7: Immuno detection by western blot of the expression of AR, H2AX and γH2AX 72 h after transfection of PC3 cells by one of the following siRNAs: Control (Cont), siAR7 or siARH2AX-1.

FIG. 8: Immuno detection of γH2AX (first column) and 53BP1 (second column) in C4-2 cells treated for 6 h with bleomycin (10 μl) or the vehicle added 72 h after transfection of cells by a control siRNA (Cont), siAR7 siRNA, or siARH2AX-1.

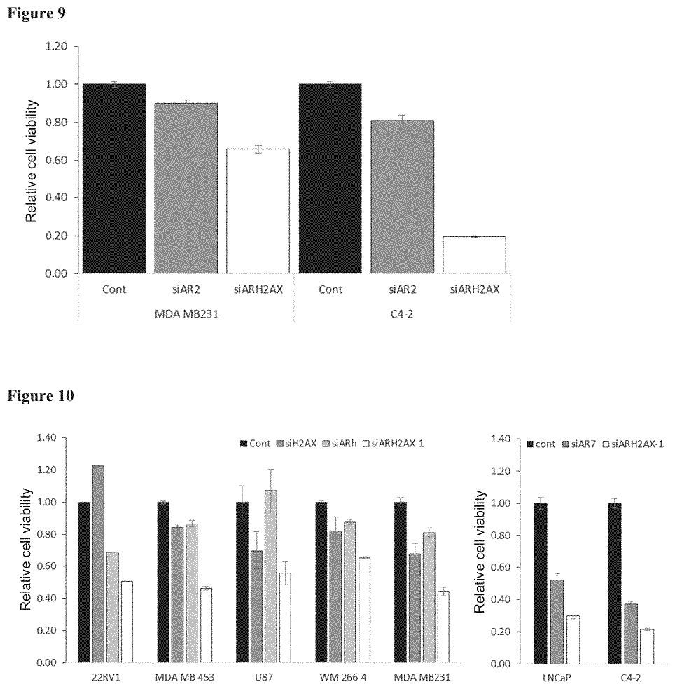

FIG. 9: Measurement of cell viability by a WST1 test of MDA MB231 or C4-2 cells 90 h after transfection of a control siRNA (black bars) of siAR2 (gray bars) or siARH2AX-1 (white bars). The measured values are related to the average of the values measured in the control condition (control siRNA, vehicle).

FIG. 10: Left panel: 22RV1, MDA MB-453, U87, WM266-4 and MDA MB231 cell viability measured by WST1 test 72 hr after transfection of a siRNA control (Cont), siARh, siH2AX or siARH2AX. Right panel: LNCaP or C4-2 cell viability measured by WST1 test 72 hr after transfection of a siRNA control (Cont), siAR7, or siARH2AX. For each line, the results are related to cell viability in the control condition.

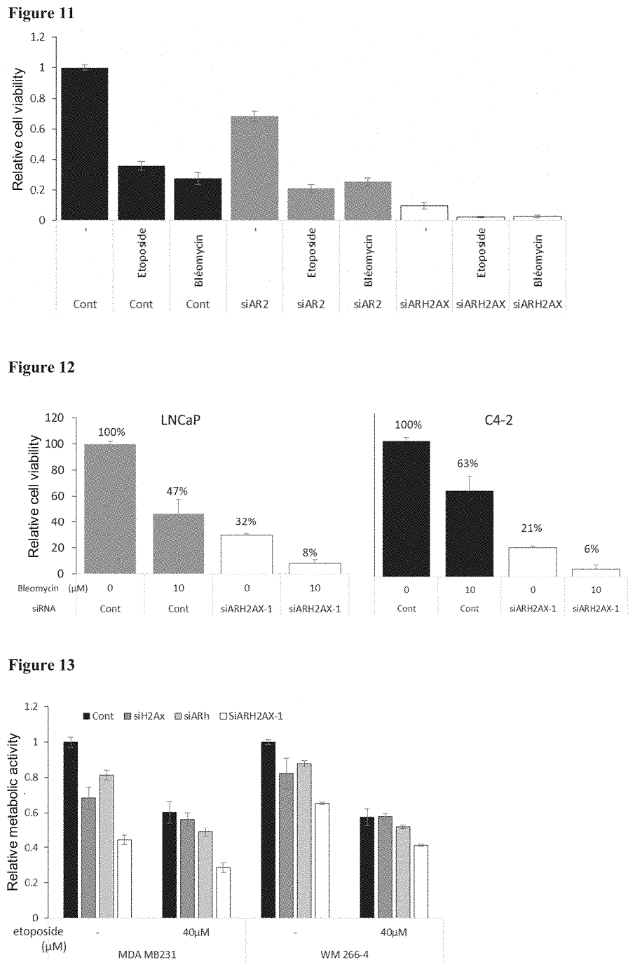

FIG. 11: Measurement of cell viability by a WST1 test of C4-2 cells transfected for 48 h by a control siRNA (black bars), siAR2 (gray bars) or siARH2AX-1 (white bars) and then incubated for 48 h in a medium containing 10 μM of bleomycin or 10 μM of etoposide or the vehicle. The measured values are related to the average of the values measured in the control condition (control siRNA, vehicle).

FIG. 12: LNCaP or C4-2 cell viability, measured by a WST1 metabolic test, 48 h after treatment with bleomycin 10 μM or by its vehicle added 24 h after transfection of the cells by a control siRNA (Cont) or by siARH2AX-1. The viability measurements of the different samples are related to the average of the values measured in the cells cultured under the control condition (siRNA control without bleomycin) considered as 100%.

FIG. 13: MDA MB231 and WM266-4 cell viability, measured by a WST1 metabolic test, 48 h after treatment with 40 μM of etoposide or by its vehicle added 24 hours after transfection of the cells by a control siRNA (Cont, black bars), siH2AX siRNA (dark gray bars), siARh siRNA (light gray bars) or siARH2AX-1 (white bars). The viability measurements of the different samples are related to the average of the values measured in the cells cultured under the control condition (siRNA control without bleomycin) considered as 100%.

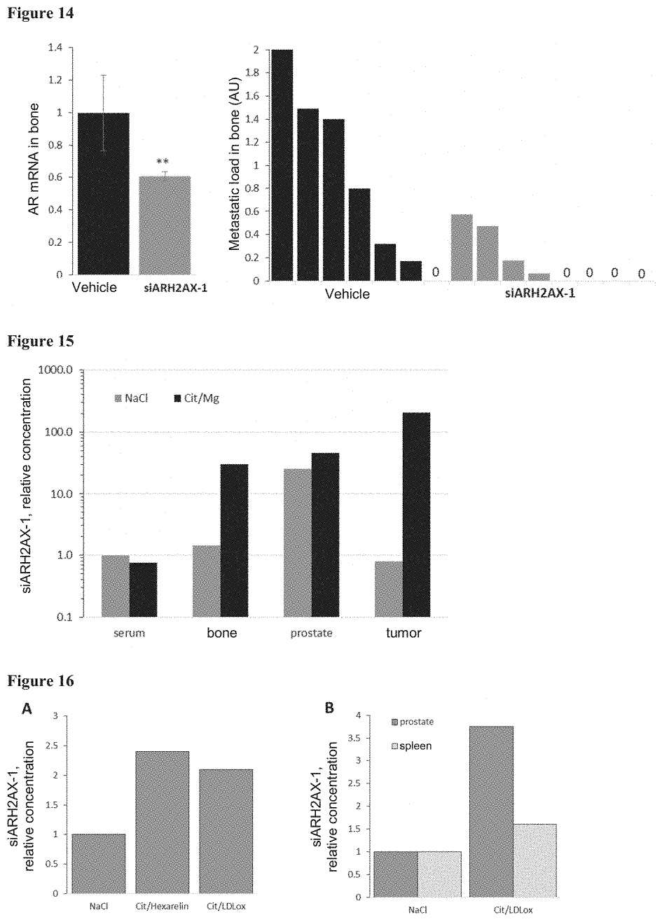

FIG. 14: Inhibition of bone metastases from prostate cancer by continuous systemic administration of siARH2AX-1.

Left panel: Expression level of human AR mRNA in nude mouse tibia carrying 22RV1 human prostate tumors in vehicle-treated mice (154 mM NaCl, black bar) or siARH2AX-1 diluted in this vehicle (gray bar) and administered subcutaneously continuously for 3 weeks (mean±SEM, n=7 values relative to the mean value of the NaCl group).

Right panel: Metastatic load in bone as measured by the expression of human HPRT mRNA in both groups of animals. Each bar represents bone metastatic load in a mouse. “0” indicates that HPRT mRNA was not detected in this animal.

FIG. 15: Quantification by RT-qPCR in serum, different organs and tumors of siARH2AX-1 administered subcutaneously continuously for 3 days, diluted either in a solution of 154 mM NaCl (gray bars) or in a citrate buffer at pH 6 containing 10 mM MgCl₂ (black bars) (mean±SEM, n=4, values referred to the mean value of the NaCl group in the serum).

FIG. 16: Effect of a composition comprising siH2AX-1 siRNA and a CD36 receptor ligand administered as a single dose on siRNA distribution in vivo. Quantification by RT-qPCR in the serum (panel A) or different organs (panel B) of siARH2AX-1 10 minutes after subcutaneous administration at the single dose of 0.12 mg/kg formulated either in a saline solution (154 mM NaCl), or in 10 mM citrate buffer pH 6 containing hexarelin, (siAR-1:hexarelin 1:1, w/w), or oxidized LDL (siAR-1:LDL oxidized 1:0.1, w/w) The mean of each group (3 mice per group) was reported as the mean of the values of the NaCl group.

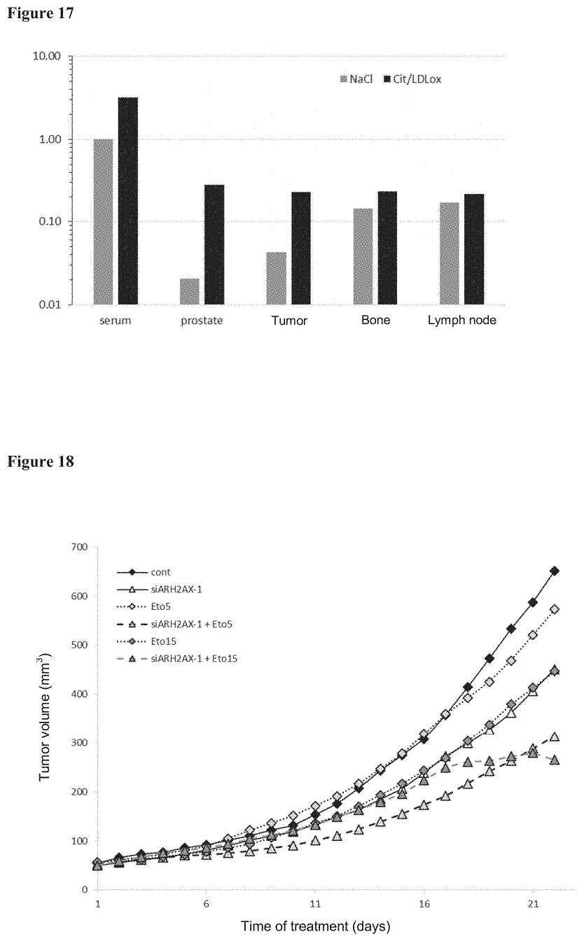

FIG. 17: Effect of a composition comprising siH2AX-1 siRNA and a CD36 receptor ligand administered systemically subcutaneously continuously on the distribution of siRNA in vivo. Osmotic pumps delivering continuously for 3 days 2 mg/kg/day of siARH2AX-1 siRNA were implanted subcutaneously in mice bearing human prostatic tumors 22RV1. The siRNA was formulated either in a saline solution (154 mM NaCl, gray bars) or in 10 mM citrate buffer at pH 6 containing 1 μM MgCl₂ and supplemented with oxidized LDL (siARH2AX-1:oxidized LDL 1:0.1, weight/weight) (black bars). siARH2AX-1 was quantified by RT-qPCR at the end of the 3 days of treatment. The mean of each group (3 mice per group) was related to the mean values in the serum of the NaCl group. The axis of ordinates indicates the concentration of siARH2AX-1 relative to that measured in the serum of the mice having received the siRNA diluted in a solution of NaCl.

FIG. 18: Volume versus time of C4-2 tumors developing by nude mice treated three times a week by the vehicle (DMSO) (black diamonds), etoposide at a dose of 5 mg/kg (gray diamonds clear), or 10 mg/kg (dark gray diamonds), or treated with continuous subcutaneous administration of siARH2AX-1 siRNA (white triangles), administered alone or in combination with etoposide at a dose of 5 (light gray triangles) or 10 (dark gray triangles) mg/kg three times a week.

### DESCRIPTION OF THE PREFERRED EMBODIMENTS

### Examples

### Example 1: Methods

1—Used siRNA

A siRNA not hybridizing with any known mRNA (siRNA Control) was used. This siRNA, Cont, is composed of the following two oligonucleotides:

SEQ ID NO 7:
5′UAGCAAUGACGAAUGCGUA[dT][dT]
SEQ ID NO 8:
5′UACGCAUUCGUCAUUGCUA[dT][dT]

Four siRNA inhibiting AR are used:
siARH2AX siRNA is composed of two oligonucleotides SEQ ID NO: 1 and SEQ ID NO: 2 as indicated in Table 1.
siAR2 siRNA, described in application PCT/FR2002/003843, is composed of the two following oligonucleotides:

SEQ ID NO 9:
GACUCAGCUGCCCCAUCCACG[dT][dT]
SEQ ID NO 10:
CGUGGAUGGGGCAGCUGAGUC[dT][dT]

Two other siRNAs hybridizing with AR mRNA but not with that of H2AX were used. siARh hybridizes with a sequence of the first exon of AR, siAR7 with a sequence of the 7° exon of AR. These siRNAs are composed of the two following oligonucleotides:

siARh:
SEQ ID NO 11:
UCCCCAAGCCCAUCGUAGA[dT][dT]
SEQ ID NO 12:
UCUACGAUGGGCUUGGGGA[dT][dT]
siAR7:
SEQ ID NO 13:
AAUGAUACGAUCGAGUUCC[dT][dT]
SEQ ID NO 14:
GGAACUCGAUCGUAUCAUU[dT][dT]

A siRNA, siH2AX, hybridizing with H2AX mRNA but not with that of AR was also used. This siRNA is composed of the two following oligonucleotides:

siH2AX:
SEQ ID NO 15:
CUGGAAUUCUGCAGCUAAC[dT][dT]
SEQ ID NO 16:
GUUAGCUGCAGAAUUCCAG[dT][dT]

2—Cell Lines, Culture and Transfection Conditions, Gene Expression Measurement

All the cells used in the following examples are human cells and express H2AX.

Table 2 indicates the origin of the lines and the relative expression level of the AR mRNA, measured by RT-qPCR normalized to the expression of cyclophilin-A and expressed in arbitrary units (22RV1=100,000).

The cells are cultured in DMEM or RPMI supplemented with 10% fetal calf serum, or as recommended by the ATCC. When indicated, they are transfected with siRNA using lipofectamine (Life Technologies), following the supplier's protocol. The number of living cells after the indicated treatments was quantified by measuring their metabolic activity by the WST1 test according to the supplier's indications (Roche).

The effect of siRNA transfection and/or treatments by different chemotherapeutic agents on gene expression was measured:
by RT-qPCR quantification of mRNAs extracted by a conventional phenol-chloroform method (Trizol). The amount of RNA was normalized by measuring the cyclophilin A-encoding mRNA which is not regulated by AR (delta delta CT method).
by Western blotting on protein extracts. The antibodies used are: AR: Santacruz, N-20 sc-816; H2AX: Merck Millipore ref 07-627; Tubulin: Sigma T6199; Actin: Merck Millipore MAB 1501; γH2AX: Merck Millipore 05-636. Immuno-detection of tubulin or actin is used to ensure that comparable amounts of protein are analyzed under the different conditions.
by indirect immunofluorescence on cells cultured on glass coverslip and fixed. The antibodies used are the following: γH2AX: Merck Millipore 05-636; 53BP1: Novus, NB 100-304.

3—Quantification of siRNA by a Modified Quantitative RT-PCR Method

To quantify a siRNA in the biological samples, the inventors have developed a reverse transcription method followed by a quantitative PCR (RT-qPCR). For each siRNA, a specific stem-loop primer, having 8 overflowing nucleotides is synthesized, the 8 nucleotides being complementary to the 8 nucleotides of the 3′ end of the siRNA guide (antisense) strand. After a reverse transcription step, the product obtained is amplified by PCR using two primers, one hybridizing with the region corresponding to the loop of the reverse transcription primer. The 12 nucleotides at 3′ of the second primer having a DNA sequence corresponding to the 12 nucleotides of the 5′ end of the newly synthesized cDNA after reverse transcription of the siRNA guide strand. Detection of the amplification is carried out continuously by the degradation of a Taqman fluorescent probe or by incorporation of SybrGreen.

A range of double stranded siRNAs, from 10³ to 10⁹ copies in the RT reaction, diluted in water is made and treated together with the samples by RT-qPCR. FIG. 1 shows schematically the RT-qPCR method and an exemplary range showing the relationship between the number of copies present in the reaction and the CT (cycle threshold or amplification threshold) obtained.

The biological samples in which siRNAs are quantified are either serum or total RNAs extracted from tissue fragments of known weight assumed to contain siRNA. These RNAs are extracted by conventional methods such as by the phenol-chloroform method (trizol extraction). After extraction, they are diluted in water.

The CT values obtained for each sample are compared with those obtained for the range. This makes it possible to calculate the number of siRNA copies present in the assayed sample. The values are then reported firstly to the amount of total transcribed reverse RNA, then to the tissue weight from which the RNAs were extracted or to the serum volume, and the final results are expressed in moles/L (M) considering that the density of all tissues is 1 g/cm³.

4—Tumor Cell Transplant in Mice

Tumors are obtained by subcutaneous injection of tumor cells into the flank of Nude mice. Only animals on which tumor uptake is found are included in the study and randomized to receive treatment or control treatment.

When indicated, Etoposide, diluted in DMSO and then adjusted to the required concentration in a 154 mM NaCl solution, is injected intraperitoneally at the indicated dose, 3 times per week.

All siRNAs are diluted in water containing 154 mM NaCl or in the indicated buffer.

When indicated, the continuous administration of siRNA is carried out by implantation of an Alzet osmotic pump subcutaneously on the back of the mice, on the opposite side to the tumor when the mice wear one. The concentration of siRNA in the pump is adjusted to deliver the indicated amount in mg/kg/day taking into account the pump flow rate indicated by the manufacturer.

The subcutaneous tumors volume is estimated by measuring with a calliper the largest (D), the smallest (d) diameter of the tumors and their height (h). The volume is calculated by the formula V=D×d×h×Pi/6.

At the end of the experiment, the animals are sacrificed, the serum, the tumors and different tissues are dissected, the extracted RNAs and the siRNAs present in these RNAs are quantified.

All the experimental protocols used have been validated by the French ethics committees and regulatory authorities. They are implemented in such a way as to limit the number of animals used and to avoid unnecessary suffering.

### Example 2: siARH2AX-1 and siARH2AX-1b siRNA of the Invention are Bispecific

The alignments shown in FIG. 2 show that the 19 siARH2AX-1 bases and the 21 siAR2 bases are perfectly complementary to the AR mRNA. This figure also shows that 17 bases out of 19 of siARH2AX-1 and 17 bases out of 21 of siAR2 hybridize with those of the H2AX-encoding mRNA. The presence of two deoxythymidines overflowing with these siRNAs has no effect on the hybridization of these siRNAs.

The siRNA nucleation region of siARH2AX-1 and siARH2AX-1b, i.e. nucleotides 2 to 8 included from the 5′ to 3′ sequence of their guide strand, (gray box in FIG. 1) is perfectly complementary (7 bases out of 7, i.e. 100%) of the H2AX mRNA. This complementarity is however only partial, from 5 bases out of 7, that is only 71% for siAR2, the nucleotides in position 2 and 3 of siAR2 not being complementary to the H2AX mRNA. This difference causes a functional difference between siARH2AX-1 and siAR2 as shown in FIG. 3. In this experiment, C4-2 tumor cells were transfected with the following siRNAs: Cont, siARH2AX-1 or siAR2. 48 hours after transfection, it is observed that siARH2AX-1 and siAR2 siRNA inhibit AR expression in a similar manner. In contrast, siAR2 siRNA whose nucleation region is only partially (71%) complementary to the H2AX mRNA is considerably less effective at inhibiting the expression of H2AX than the siARH2AX-1 siRNA whose nucleation region is perfectly complementary to the H2AX mRNA.

In FIGS. 4, 5, 6 and 7, different cell lines were transfected with siARH2AX-1 and two other siRNAs targeting a sequence of exon 1 (siARh), or exon 7 (siAR7) of AR, respectively, but not targeting H2AX, or even by a siRNA targeting H2AX but not AR.

In the experiment shown in FIG. 4, the LNCaP or C4-2 tumor cells were transfected with the following siRNAs: Cont, siAR7 or siARH2AX-1. 72 hours after transfection, the analysis of the AR-, Antigen-specific Prostate-, or PSA-encoding mRNAs, which is a gene regulated by the androgen receptor, and those encoding H2AX shows that in both lines, siAR7 and siARH2AX-1 inhibit the expression of AR and PSA, but that only siARH2AX-1 also inhibits the expression of H2AX, to more than 90%.

Similarly, in the experiment shown in FIG. 5, LNCaP or MDA MB-453 tumor cells were transfected with the following siRNAs: Cont, siH2AX, siARh, siARH2AX-1. In both lines, siARh inhibits AR but not H2AX, siH2AX inhibits H2AX but not AR. The siARH2AX-1 siRNA alone inhibits the expression of the two AR- and H2AX-encoding mRNAs.

In the experiment shown in FIG. 6, the protein expression of AR and H2AX was measured 72 h after transfection of C4-2 cells by the following siRNAs: Cont, siARH2AX-1, siARh, siH2AX, and a mixture (1:1) of both siARh and siH2AX siRNA. siARh inhibits AR but not H2AX, siH2AX inhibits H2AX but not AR, the mixture of both siRNA inhibits AR and H2AX. The siARH2AX-1 siRNA alone inhibits the expression of both AR and H2AX proteins.

In the experiment shown in FIG. 7, the PC3 tumor cells, which express weakly AR, were transfected with the following siRNAs: Cont, siAR7 or siARH2AX-1. 72 hours after transfection, the expression of AR and H2AX proteins was measured. It is observed that the expression of H2AX is inhibited by siARH2AX-1 but not by siAR7. Inhibition of H2AX is accompanied by inhibition of its γH2AX phosphorylated form.

Taken together, these results show that only siARH2AX-1 siRNA has the ability to inhibit both AR and H2AX at the same time. siARH2AX-1 siRNA is much more effective than siAR2 siRNA in inhibiting H2AX expression. A difference of two bases in the nucleation region of these two siRNAs profoundly modifies their ability to inhibit H2AX.

Inhibition of H2AX by siARH2AX-1 occurs in cells expressing AR strongly (LNCaP, C4-2), intermediate (MDA MB253) or weakly (PC3).

### Example 3: Inhibition of H2AX Prevents the Recruitment of 53BP1 on Bleomycin-Induced DNA Lesions and Decreases Cell Viability

In the experiment shown in FIG. 8, C4-2 cells were transfected with a control or siARH2AX-1 siRNA. 72 hours after transfection, the cells were then treated for 6 hours with 10 μl of bleomycin or the corresponding vehicle.

It is observed that in the control condition (Cont siRNA, absence of bleomycin), γH2AX is detectable but weakly expressed, whereas the expression of 53BP1 is predominantly diffuse. The bleomycin treatment strongly increases the expression of γH2AX whereas the 53BP1 labeling becomes punctiforme, translating its recruitment via γH2AX on DNA lesions induced by bleomycin.

In siAR7-transfected cells, bleomycin induces an increase in γH2AX labeling and recruitment of 53BP1.

In cells transfected with siARH2AX-1, the expression of H2AX is very small compared to the control condition whereas the expression of 53BP1 is comparable. After treatment with bleomycin, siARH2AX-1 transfected cells showed no increase H2AX or recruitment of 53BP1 to focal points of repairing DNA lesions.

It is concluded from this experiment that siARH2AX siRNA inhibits DNA repair.

### Example 4: siARH2AX-1 siRNA Reduces Cell Viability Regardless of the Level of AR Expression

In the experiment described in FIG. 9, it is observed that 90 hours after transfection of MDA MB231 cells which express weakly AR, or of C4-2 cells, the viability of siARH2AX siRNA-transfected cells is significantly reduced in the two lines then only marginally in cells transfected with siAR2siRNA.

In the experiment shown in FIG. 10, in the left panel, the cells were transfected with a Control (Cont), siH2AX, siARh, or siARH2AX-1 siRNA and in the right panel with a Control, siAR7 or siARH2AX-1 siRNA. The siARh and siAR7 siRNA inhibit the proliferation of cells that strongly express AR (22RV1, C4-2, LNCaP) but not other lines expressing intermediate or low levels of AR (MDA MB 453, MDA MB 231, WM 266-4, U87).

In contrast, siARH2AX-1 siRNA inhibits the proliferation of all cell lines, regardless of the level of AR expression, and regardless of the effect on their proliferation of AR inhibition by the siARh siRNA that does not inhibit H2AX whereas siH2AX siRNA that only inhibits H2AX expression without affecting AR has little effect on cell proliferation, regardless of lineage.

It is concluded from these experiments that siARH2AX-1 siRNA alone inhibits cell proliferation, whether it depends or does not depend on AR expression and that AR is expressed very strongly at an intermediate level, or very weakly in these cells.

### Example 5: The Effects of siARH2AX-1 siRNA and DNA Damage Inducing Agents are Potentiated, Regardless of the Level of AR Expression

In the experiment described in FIG. 11, 48 hours after transfection of C4-2 cells by cont, siAR2 or siARH2AX-1 siRNA, the cells were treated with a vehicle or with etoposide (10 μM) or bleomycin (10 μM), chemotherapy agents inducing DNA damage. 48 hours after addition of the chemotherapy agents, it is observed that the combination of siAR2 siRNA with one or other of these agents does not produce a significantly greater viability-inhibiting effect than that measured by the chemotherapeutic agent alone. In contrast, the effects of siARH2AX-1 and either of the chemotherapeutic agents potentiate to inhibit cell viability. In the experiment described in FIG. 12, 48 h after transfection of C4-2 cells by cont or siARH2AX-1 siRNA, the cells were treated with a vehicle or with bleomycin (10 μM). It is observed that the combination of siARH2AX-1 with bleomycin treatment in LNCaP cells produces effects approximately twice as inhibitory to cell survival as would be expected from the addition of the effects of the two separate treatments, showing potentiation of these two treatments.

In the experiment shown in FIG. 13, MDA MB 231 or WM266-4 cells that express weakly AR were transfected with a control (Cont), siH2AX, siARh, or siARH2AX-1 siRNA. 24 hours after transfection, the cells were treated with 40 μl of etoposide or by the vehicle, and the cell viability, relative to that of the cells under controlled conditions, was measured by a WST1 test 48 h later. It is observed that in both lines, etoposide inhibits cell proliferation. When this treatment is associated with inhibition of H2AX by siH2AX, or with inhibition of AR by siARh siRNA, there is no amplification of the effects of etoposide on proliferation. On the other hand, etoposide and siARH2AX-1 treatments potentiate when combined.

From these experiments it is concluded that it is advantageous to combine a DNA damage-inducing treatment with siARH2AX-1 to inhibit tumor cell proliferation, including in cells expressing low levels of AR. siRH2AX-1 siRNA is much more effective than siAR2 siRNA in inhibiting H2AX expression and cell viability, including in cells expressing low AR. In addition, siARH2AX-1, but not siAR2, potentiates the inhibitory effects of viability produced by chemotherapies.

### Example 6: Administration of siARH2AX-1 to Tumor-Bearing Mice Inhibits Metastatic Spread

In the experiment shown in FIG. 14, 22RV1 cells were implanted in Nude mice. Once the tumor was detected, Alzet pumps administering either 0.2 mg/kg/day siARH2AX-1 siRNA formulated in saline (154 mM NaCl), or the vehicle alone, were implanted for 3 weeks. At the end of treatment, the bones (tibia) were recovered to quantify siARH2AX-1, and the androgen receptor- and the HPRT-encoding mRNAs of human origin.

The continuous subcutaneously administration of siARH2AX-1 siRNA to mice with human prostatic tumors 22RV1 to inhibit the expression of the androgen receptor in the bones of mice (FIG. 14 left panel). This inhibition is accompanied by a decrease in the number of mice spontaneously developing bone metastases of these tumors and a decrease in the size of these tumors, evaluated by the level of expression of a human mRNA (HRPT) in the cells. bone (FIG. 14 right panel).

The continuous subcutaneous systemic administration of siARH2AX-1 siRNA allows it to be delivered in the metastases of a cancer developing in bone, to inhibit the expression of the target siRNA gene in the bone and to limit the implantation and/or the development of metastases.

### Example 7: Improving the Distribution of an siRNA by its Formulation

In the experiment shown in FIG. 15, osmotic pumps continuously delivering siARH2AX-1 at the dose of 2 mg/kg/day diluted either in saline solution (154 mM NaCl) or in solution in a citrate buffer at pH 6 containing 10 mM of MgCl₂ (Cit/mg), were implanted subcutaneously in tumor-bearing mice. After 3 days, siARH2AX-1 was quantified by RT-qPCR in serum, various organs, and tumors. It is found that compared to the saline formulation, the citrate buffer formulation at pH 6 supplemented with 10 mM MgCl₂ significantly increases the concentration of siARH2AX-1 in tissues and tumors.

The CD36 receptor is expressed to the outer membrane of endothelial cells of many tumor cells and other cell types such as macrophages. This CD36 receptor binds different classes of ligands, such as oxidized LDLs or hexarelin peptide.

In the experiment shown in FIG. 16, siARH2AX-1 siRNA, at the dose of 0.12 mg/kg, formulated in a solution of 154 mM NaCl (control group, denoted NaCl) or in a 10 mM citrate buffer pH 6 Addition of 0.02 mM hexarelin peptide (Cit/Hexarelin) or 30 nM oxidized LDL (Cit/LDL) was administered as a bolus subcutaneous injection to groups of adult mice. 20 minutes after injection, the mean concentration of the siRNA measured in the serum (panel A) or tissues (panel B) of these different groups of mice (4 mice per group), relative to the value of the average of the control group, shows that the addition of hexarelin or oxidized LDL increases the concentration of siRNA in serum and tissues.

In the experiment shown in FIG. 17, an osmotic pump was used to administer to mice carrying 22RV1 human prostatic tumors subcutaneously and continuously for 3 days siARH2AX-1 siRNA at a dose of 2 mg/kg/day. The siRNA was formulated either in 154 mM NaCl solution, or in 10 mM citrate buffer pH 6 containing oxidized LDL (siARH2AX-1:LDLox ratio 1:0.1, weight:weight). The concentration of siARH2AX-1 was measured after 3 days of treatment in serum, different organs and tumors. It is found that the addition of oxidized LDL increases the concentration of siARH2AX-1 in serum, different organs and tumors.

### Example 8: The Combination of siARH2AX-1 and a DNA Damage Inducing Agent Potentiates the Effect of Each of these Separately Administered Treatments on Tumor Development

In the experiment shown in FIG. 18, an osmotic pump was implanted into 22RV1 human prostatic tumor-bearing mice for subcutaneous and continuous administration for 22 days from day 1 of siARH2AX-1 siRNA to dose of 2 mg/kg/day formulated in 10 mM citrate buffer pH 6 containing 10 mM MgCl₂, or the vehicle alone. Two days after implantation of the pumps, the mice received an intraperitoneal injection of etoposide at a dose of 5 or 15 mg/kg or the vehicle and this treatment was repeated twice a week until sacrifice of the mice. It is observed that etoposide at 5 mg/kg only slightly inhibits tumor development. The dose of 15 mg/kg produces an inhibition but is accompanied in several animals treated with a weight loss indicating a toxicity of this treatment. siARH2AX-1 siRNA is as effective as the high dose of etoposide without causing weight loss.

The combination of treatment with etoposide and siARH2AX-1 potentiates the effect of these two treatments taken separately.

It is concluded from this experiment that siARH2AX-1 siRNA effectively inhibits tumor development, with no significant toxic effect in animals, and that the combination of this treatment with low, non-toxic doses of an agent inducing DNA amplifies tumor growth inhibition.

1-13. (canceled)

14. A method for the prevention and/or treatment of a cancer comprising the administration of a composition comprising at least one bispecific siRNA hybridizing in whole or in part with both an androgen receptor-encoding mRNA and an H2AX-encoding mRNA, such that such hybridization induces the degradation of these mRNAs or inhibits their translation, said siRNA being selected from siRNA: siARH2AX-1, SEQ ID NO: 1 and SEQ ID NO: 2 or siARH2AX-1b, SEQ ID NO: 3 and SEQ ID NO: 4, wherein said siRNA is used in combination with a therapy resulting in DNA damage or preventing its repair.

15. The method of claim 14, wherein said therapy resulting in DNA damage is selected from radiotherapy and/or chemotherapy.

16. The method of claim 14, wherein said chemotherapy resulting in DNA damage or preventing its repair is performed using at least one agent selected from: alkylating agents, anti-metabolite agents, cytotoxic antibiotics, topoisomerase I inhibitors, topoisomerase II inhibitors, anti-tumor antibiotics, genotoxic agents, PARP inhibitors, and is chosen in particular from: Arabinosylcytosine, Bleomycin, Busulfan, Capecitabine, Carboplatin, Carmustine, Chlorambucil, Cisplatin, Cladribine, Cyclophosphamide, Dacarbazine, Daunorubicin, Doxorubicin, Epirubicin, Etoposide, Floxuridine, Fludarabine, Fluorouracil and in particular 5-Fluorouracil, Gemcitabine, Hydroxyurea, Idarubicin, Ifosfamide, Iniparib, Irinotecan, Lomustine, Mechlorethamine, Melphalan, Mercaptopurine and in particular 6-Mercaptopurine, Methotrexate, Mitomycin C, Mitoxantrone HCl, Mustine, Niraparib, Olaparib, Oxaliplatin, Procarbazine, Rubitecan, Rucaparib, Streptozocin, Talazopanib, Thioguanine and in particular 6-Thioguanine, Topotecan, Veliparib, Actinomycin D, Amsacrine, Anthracyclines, Camptothecin, Epipodophyllotoxin, Plicamycin, Temozolomide, Vincristine, Vinblastine, Vinorelbine, Paclitaxel, Docetaxel, Cabazitaxel, Taxanes, Epothilones.

17. The method of claim 14, wherein the cancer is adrenocortical, esophageal, gastric, basal cells, thyroid or uterine, astrocytoma, glioblastoma, oligodendroglioma, meningioma, thymoma, lymphoma, melanoma, or non-melanoma skin cancer, leukemia, mesothelioma, cholangiocarcinoma, myeloma, gastrointestinal cancer, bladder, breast, cervical, head and neck, non-small cell lung, ovary, pancreas, prostate, testis, thymus, kidney, salivary gland, endometrium, anus, colon, appendix, mouth, bronchi and/or upper airways, bile duct, nasal and paranasal cavity, brain, heart, stomach, liver, throat, tongue, lips, nasopharynx, esophagus, bones, parathyroid, penis, pleura, lung, rectum, adrenal gland, urethra, vagina, gallbladder, vulva, colon adenocarcinoma, rectum, desmoid tumor, nasopharyngeal fibroma, sarcoma, osteosarcoma, leimyosarcoma, chondrosarcoma, liposarcoma, rhabdomyosarcoma, pheochromocytoma or metastasis of these cancers developing in other organs.

18. The method of claim 14, wherein said composition is formulated for a local, loco-regional, and in particular intratumoral or systemic administration mode, in single or repeated doses, intermittently or continuously, and in particular in which said systemic administration mode is selected from the subcutaneous, intravenous, intraperitoneal, intramuscular, intradermal, transdermal, intranasal, intravaginal, intrarectal, sublingual, intrathecal, intracerebral, and oral administration mode, and in particular a continuous and subcutaneous administration mode.

19. The method of claim 14, wherein said bispecific siRNA is in a buffer solution at acidic pH, in particular in a citrate or histidine buffer.

20. The method of claim 14, wherein said bispecific siRNA is in a buffer solution at acidic pH, in particular in a citrate or histidine buffer and
wherein said bispecific siRNA is in a buffer solution at acidic pH, added with inorganic or organic salts, in particular salt whose cation is chosen from polyamines, in particular chosen from spermine, spermidine or putrescine or in particular salt whose cation is chosen from metal cations, in particular chosen from salts of zinc, cobalt, copper, manganese, calcium, magnesium or iron, in particular of manganese, zinc, magnesium, alone or in two to two or three to three combination.

21. The method of claim 14, wherein said siRNA is formulated for a siRNA administration mode at a therapeutically effective dose, and in particular at doses of 0.005 mg/kg/day to 30 mg/kg/day, more particularly 0.01 mg/kg/day to 10 mg/kg/day.

22. The method of claim 14, wherein said composition does not contain a vectorization agent or addressing molecule.

23. The method of claim 14, wherein said composition does not contain a vectorization agent and contains an addressing molecule, in particular a CD36 ligand, and in particular oxidized LDLs or Hexarelin.

24. The method of claim 14, wherein said composition contains a vectorization agent and does not contain an addressing molecule.

25. The method of claim 14, wherein said composition contains a vectorization agent and contains an addressing molecule, in particular a CD36 ligand, and in particular oxidized LDLs or Hexarelin,

26. The method of claim 14, wherein said at least one bispecific siRNA is devoid of chemical modification or present chemical modification.
